SEQUENCE LISTING (42 sequences)

SEQ ID NO: 1 - protein, 79 aa, Homo sapiens
  MISC_FEATURE (2)..(2): Xaa reflects Thr or Ser variants
GXHSXXPRPAAVPVPXXXQPXPAHPVLSFLXPSWDXVSAFYSLPLAPLSPTSVXISPVSVGRGXDPDAHVAVXLSRYEG

SEQ ID NO: 2 - protein, 419 aa, Homo sapiens
  MISC_FEATURE (342)..(342): Xaa reflects Thr or Ser variants
MELAALCRWGLLLALLPPGAASTQVCTGTDCKLRLPASPETHLDMLRHLYQGCQVVQGNLELTYLPTNASLSFLQDIQEVQGYVLCAHNQVRQVPLQRLRIVRGTQLFEDNYALAVLDNGDPLRRTTPVTGASPGGLRELQLRSLTECLKGGVLIQRNPQLCYQDTILWKDIFHKNNQLALTLIDTNRSRACHPCSPCCKGSRCWGESSEDCQSLTRTVCAGGCARCKGPLPTDCCHEQCAAGCTGPKHSDCLACLHFNHSGICELHCPALVTYNTDTFESCPNPEGRYTFGASCVTACPYNKLSTDVGSCTLVCPLHNQEVTAEDGTQRCEKCSKPCARGXHSXXPRPAAVPVPXXXQPXPAHPVLSFLXPSWDXVSAFYSLPLAPLDPTSVXISPVSVGRGXDPDAHVAVXLSRYEG

SEQ ID NO: 3 - protein, 79 aa, artificial sequence
  ECDIIIA domain non-binding mutant (Arg to Ile mutation at residue 31)
GXHSXXPRPAAVPVPXRXQPXPAHPVLSFLIPSWDXVSAFYSLPLAPLSPTSVXISPVSVGRGXDPDAHVAVXLSRYEG

SEQ ID NO: 4 - protein, 419 aa, artificial sequence
  Herstatin; receptor non-binding mutant (Arg to Ile mutation at residue 371)
MELAALCRWGLLLALLPPGAASTQVCTGTDCKLRLPASPETHLDMLRHLYQGCQVVQGNLELTYLPTNASLSFLQDIQEVQGYVLCAHNQVRQVPLQRLRIVRGTQLFEDNYALAVLDNGDPLNNTTPVTGASPGGLRELQLRSLTECLKGGVLIQRNPQLCYQDTILWKDIFHKNNQLALTLIDTNRSRACHPCSPCCKGSRCWGESSEDCQSLTRTVCAGGCARCKGPLPTDCCHEQCAAGCTGPKHSDCLACLHFNHSGICELHCPALVTYNTDTFESCPNPEGRYTFGASCVTACPYNKLSTDVGSCTLVCPLHNQEVTAEDGTQRCEKCSKPCARGXHSXXPRPAAVPVPXRXQPXPAHPVLSFLIPSWDXVSAFYSLPLAPLDPTSVXISPVSVGRGXDPDAHVAVXLSRYEG

SEQ ID NO: 5 - DNA, 5616 bp, Homo sapiens
  CDS (247)..(3879): HER-1 coding sequence
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 6 - protein, 1210 aa, Homo sapiens [sequence omitted]

SEQ ID NO: 7 - DNA, 4815 bp, Homo sapiens
  CDS (247)..(3078): delta EGFR coding sequence [represents in- frame deletion of 801 bp (275-1027) of the ECD of EGFR corresponding to exons 2-7 of the gene]
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagtggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 8 - protein, 943 aa, Homo sapiens
MRPSGTAGAVDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 9 - DNA, 4530 bp, Homo sapiens
  CDS (151)..(3918): HER-2 coding sequence
aattctcgagctcgtcgaccggtcgacgagctcgagggtcgacgagctcgagggcgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcgtctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaagggccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagacgaaataaagacccaggggagaatgggtgttgtatggggaggcaagtgtggggggtccttctccacacccactttgtccatttgcaaatatattttggaaaac

SEQ ID NO: 10 - protein, 1255 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIVSAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERAKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 11 - DNA, 4975 bp, Homo sapiens
  CDS (199)..(4227)
ctctcacacacacacacccctcccctgccatccctccccggactccggctccggctccgattgcaatttgcaacctccgctgccgtcgccgcagcagccaccaattcgccagcggttcaggtggctcttgcctcgatgtcctagcctaggggcccccgggccggacttggctgggctcccttcaccctctgcggagtcatgagggcgaacgacgctctgcaggtgctgggcttgcttttcagcctggcccggggctccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttctcctgccacccggaatgccaacccatggggggcactgccacatgcaatggctcgggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtgtaaaggaccagagcttcaagactgtttaggacaaacactggtgctgatcggcaaaacccatctgacaatggctttgacagtgatagcaggattggtagtgattttcatgatgctgggcggcacttttctctactggcgtgggcgccggattcagaataaaagggctatgaggcgatacttggaacggggtgagagcatagagcctctggaccccagtgagaaggctaacaaagtcttggccagaatcttcaaagagacagagctaaggaagcttaaagtgcttggctcgggtgtctttggaactgtgcacaaaggagtgtggatccctgagggtgaatcaatcaagattccagtctgcattaaagtcattgaggacaagagtggacggcagagttttcaagctgtgacagatcatatgctggccattggcagcctggaccatgcccacattgtaaggctgctgggactatgcccagggtcatctctgcagcttgtcactcaatatttgcctctgggttctctgctggatcatgtgagacaacaccggggggcactggggccacagctgctgctcaactggggagtacaaattgccaagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaagactccaattaagtggatggcccttgagagtatccactttgggaaatacacacaccagagtgatgtctggagctatggtgtgacagtttgggagttgatgaccttcggggcagagccctatgcagggctacgattggctgaagtaccagacctgctagagaagggggagcggttggcacagccccagatctgcacaattgatgtctacatggtgatggtcaagtgttggatgattgatgagaacattcgcccaacctttaaagaactagccaatgagttcaccaggatggcccgagacccaccacggtatctggtcataaagagagagagtgggcctggaatagcccctgggccagagccccatggtctgacaaacaagaagctagaggaagtagagctggagccagaactagacctagacctagacttggaagcagaggaggacaacctggcaaccaccacactgggctccgccctcagcctaccagttggaacacttaatcggccacgtgggagccagagccttttaagtccatcatctggatacatgcccatgaaccagggtaatcttggggggtcttgccaggagtctgcagtttctgggagcagtgaacggtgcccccgtccagtctctctacacccaatgccacggggatgcctggcatcagagtcatcagaggggcatgtaacaggctctgaggctgagctccaggagaaagtgtcaatgtgtagaagccggagcaggagccggagcccacggccacgcggagatagcgcctaccattcccagcgccacagtctgctgactcctgttaccccactctccccacccgggttagaggaagaggatgtcaacggttatgtcatgccagatacacacctcaaaggtactccctcctcccgggaaggcaccctttcttcagtgggtctcagttctgtcctgggtactgaagaagaagatgaagatgaggagtatgaatacatgaaccggaggagaaggcacagtccacctcatccccctaggccaagttcccttgaggagctgggttatgagtacatggatgtggggtcagacctcagtgcctctctgggcagcacacagagttgcccactccaccctgtacccatcatgcccactgcaggcacaactccagatgaagactatgaatatatgaatcggcaacgagatggaggtggtcctgggggtgattatgcagccatgggggcctgcccagcatctgagcaagggtatgaagagatgagagcttttcaggggcctggacatcaggccccccatgtccattatgcccgcctaaaaactctacgtagcttagaggctacagactctgcctttgataaccctgattactggcatagcaggcttttccccaaggctaatgcccagagaacgtaactcctgctccctgtggcactcagggagcatttaatggcagctagtgcctttagagggtaccgtcttctccctattccctctctctcccaggtcccagccccttttccccagtcccagacaattccattcaatctttggaggcttttaaacattttgacacaaaattcttatggtatgtagccagctgtgcactttcttctctttcccaaccccaggaaaggttttccttattttgtgtgctttcccagtcccattcctcagcttcttcacaggcactcctggagatatgaaggattactctccatatcccttcctctcaggctcttgactacttggaactaggctcttatgtgtgcctttgtttcccatcagactgtcaagaagaggaaagggaggaaacctagcagaggaaagtgtaattttggtttatgactcttaaccccctagaaagacagaagcttaaaatctgtgaagaaagaggttaggagtagatattgattactatcataattcagcacttaactatgagccaggcatcatactaaacttcacctacattatctcacttagtcctttatcatccttaaaacaattctgtgacatacatattatctcattttacacaaagggaagtcgggcatggtggctcatgcctgtaatctcagcactttgggaggctgaggcagaaggattacctgaggcaaggagtttgagaccagcttagccaacatagtaagacccccatctc

SEQ ID NO: 12 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMGGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGGSCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 13 - DNA, 4975 bp, Homo sapiens
  CDS (199)..(4227): HER-3 mutant coding sequence
ctctcacacacacacacccctcccctgccatccctccccggactccggctccggctccgattgcaatttgcaacctccgctgccgtcgccgcagcagccaccaattcgccagcggttcaggtggctcttgcctcgatgtcctagcctaggggcccccgggccggacttggctgggctcccttcaccctctgcggagtcatgagggcgaacgacgctctgcaggtgctgggcttgcttttcagcctggcccggggctccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttctcctgccacccggaatgccaacccatggagggcactgccacatgcaatggctcgggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtgtaaaggaccagagcttcaagactgtttaggacaaacactggtgctgatcggcaaaacccatctgacaatggctttgacagtgatagcaggattggtagtgattttcatgatgctgggcggcacttttctctactggcgtgggcgccggattcagaataaaagggctatgaggcgatacttggaacggggtgagagcatagagcctctggaccccagtgagaaggctaacaaagtcttggccagaatcttcaaagagacagagctaaggaagcttaaagtgcttggctcgggtgtctttggaactgtgcacaaaggagtgtggatccctgagggtgaatcaatcaagattccagtctgcattaaagtcattgaggacaagagtggacggcagagttttcaagctgtgacagatcatatgctggccattggcagcctggaccatgcccacattgtaaggctgctgggactatgcccagggtcatctctgcagcttgtcactcaatatttgcctctgggttctctgctggatcatgtgagacaacaccggggggcactggggccacagctgctgctcaactggggagtacaaattgccaagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaagactccaattaagtggatggcccttgagagtatccactttgggaaatacacacaccagagtgatgtctggagctatggtgtgacagtttgggagttgatgaccttcggggcagagccctatgcagggctacgattggctgaagtaccagacctgctagagaagggggagcggttggcacagccccagatctgcacaattgatgtctacatggtgatggtcaagtgttggatgattgatgagaacattcgcccaacctttaaagaactagccaatgagttcaccaggatggcccgagacccaccacggtatctggtcataaagagagagagtgggcctggaatagcccctgggccagagccccatggtctgacaaacaagaagctagaggaagtagagctggagccagaactagacctagacctagacttggaagcagaggaggacaacctggcaaccaccacactgggctccgccctcagcctaccagttggaacacttaatcggccacgtgggagccagagccttttaagtccatcatctggatacatgcccatgaaccagggtaatcttggggggtcttgccaggagtctgcagtttctgggagcagtgaacggtgcccccgtccagtctctctacacccaatgccacggggatgcctggcatcagagtcatcagaggggcatgtaacaggctctgaggctgagctccaggagaaagtgtcaatgtgtagaagccggagcaggagccggagcccacggccacgcggagatagcgcctaccattcccagcgccacagtctgctgactcctgttaccccactctccccacccgggttagaggaagaggatgtcaacggttatgtcatgccagatacacacctcaaaggtactccctcctcccgggaaggcaccctttcttcagtgggtctcagttctgtcctgggtactgaagaagaagatgaagatgaggagtatgaatacatgaaccggaggagaaggcacagtccacctcatccccctaggccaagttcccttgaggagctgggttatgagtacatggatgtggggtcagacctcagtgcctctctgggcagcacacagagttgcccactccaccctgtacccatcatgcccactgcaggcacaactccagatgaagactatgaatatatgaatcggcaacgagatggaggtggtcctgggggtgattatgcagccatgggggcctgcccagcatctgagcaagggtatgaagagatgagagcttttcaggggcctggacatcaggccccccatgtccattatgcccgcctaaaaactctacgtagcttagaggctacagactctgcctttgataaccctgattactggcatagcaggcttttccccaaggctaatgcccagagaacgtaactcctgctccctgtggcactcagggagcatttaatggcagctagtgcctttagagggtaccgtcttctccctattccctctctctcccaggtcccagccccttttccccagtcccagacaattccattcaatctttggaggcttttaaacattttgacacaaaattcttatggtatgtagccagctgtgcactttcttctctttcccaaccccaggaaaggttttccttattttgtgtgctttcccagtcccattcctcagcttcttcacaggcactcctggagatatgaaggattactctccatatcccttcctctcaggctcttgactacttggaactaggctcttatgtgtgcctttgtttcccatcagactgtcaagaagaggaaagggaggaaacctagcagaggaaagtgtaattttggtttatgactcttaaccccctagaaagacagaagcttaaaatctgtgaagaaagaggttaggagtagatattgattactatcataattcagcacttaactatgagccaggcatcatactaaacttcacctacattatctcacttagtcctttatcatccttaaaacaattctgtgacatacatattatctcattttacacaaagggaagtcgggcatggtggctcatgcctgtaatctcagcactttgggaggctgaggcagaaggattacctgaggcaaggagtttgagaccagcttagccaacatagtaagacccccatctc

SEQ ID NO: 14 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGGSCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 15 - DNA, 5484 bp, Homo sapiens
  CDS (34)..(3960): HER-4 coding sequence
aattgtcagcacgggatctgagacttccaaaaaatgaagccggcgacaggactttgggtctgggtgagccttctcgtggcggcggggaccgtccagcccagcgattctcagtcagtgtgtgcaggaacggagaataaactgagctctctctctgacctggaacagcagtaccgagccttgcgcaagtactatgaaaactgtgaggttgtcatgggcaacctggagataaccagcattgagcacaaccgggacctctccttcctgcggtctgttcgagaagtcacaggctacgtgttagtggctcttaatcagtttcgttacctgcctctggagaatttacgcattattcgtgggacaaaactttatgaggatcgatatgccttggcaatatttttaaactacagaaaagatggaaactttggacttcaagaacttggattaaagaacttgacagaaatcctaaatggtggagtctatgtagaccagaacaaattcctttgttatgcagacaccattcattggcaagatattgttcggaacccatggccttccaacttgactcttgtgtcaacaaatggtagttcaggatgtggacgttgccataagtcctgtactggccgttgctggggacccacagaaaatcattgccagactttgacaaggacggtgtgtgcagaacaatgtgacggcagatgctacggaccttacgtcagtgactgctgccatcgagaatgtgctggaggctgctcaggacctaaggacacagactgctttgcctgcatgaatttcaatgacagtggagcatgtgttactcagtgtccccaaacctttgtctacaatccaaccacctttcaactggagcacaatttcaatgcaaagtacacatatggagcattctgtgtcaagaaatgtccacataactttgtggtagattccagttcttgtgtgcgtgcctgccctagttccaagatggaagtagaagaaaatgggattaaaatgtgtaaaccttgcactgacatttgcccaaaagcttgtgatggcattggcacaggatcattgatgtcagctcagactgtggattccagtaacattgacaaattcataaactgtaccaagatcaatgggaatttgatctttctagtcactggtattcatggggacccttacaatgcaattgaagccatagacccagagaaactgaacgtctttcggacagtcagagagataacaggtttcctgaacatacagtcatggccaccaaacatgactgacttcagtgttttttctaacctggtgaccattggtggaagagtactctatagtggcctgtccttgcttatcctcaagcaacagggcatcacctctctacagttccagtccctgaaggaaatcagcgcaggaaacatctatattactgacaacagcaacctgtgttattatcataccattaactggacaacactcttcagcacaatcaaccagagaatagtaatccgggacaacagaaaagctgaaaattgtactgctgaaggaatggtgtgcaaccatctgtgttccagtgatggctgttggggacctgggccagaccaatgtctgtcgtgtcgccgcttcagtagaggaaggatctgcatagagtcttgtaacctctatgatggtgaatttcgggagtttgagaatggctccatctgtgtggagtgtgacccccagtgtgagaagatggaagatggcctcctcacatgccatggaccgggtcctgacaactgtacaaagtgctctcattttaaagatggcccaaactgtgtggaaaaatgtccagatggcttacagggggcaaacagtttcattttcaagtatgctgatccagatcgggagtgccacccatgccatccaaactgcacccaagggtgtaacggtcccactagtcatgactgcatttactacccatggacgggccattccactttaccacaacatgctagaactcccctgattgcagctggagtaattggtgggctcttcattctggtcattgtgggtctgacatttgctgtttatgttagaaggaagagcatcaaaaagaaaagagccttgagaagattcttggaaacagagttggtggaaccattaactcccagtggcacagcacccaatcaagctcaacttcgtattttgaaagaaactgagctgaagagggtaaaagtccttggctcaggtgcttttggaacggtttataaaggtatttgggtacctgaaggagaaactgtgaagattcctgtggctattaagattcttaatgagacaactggtcccaaggcaaatgtggagttcatggatgaagctctgatcatggcaagtatggatcatccacacctagtccggttgctgggtgtgtgtctgagcccaaccatccagctggttactcaacttatgccccatggctgcctgttggagtatgtccacgagcacaaggataacattggatcacaactgctgcttaactggtgtgtccagatagctaagggaatgatgtacctggaagaaagacgactcgttcatcgggatttggcagcccgtaatgtcttagtgaaatctccaaaccatgtgaaaatcacagattttgggctagccagactcttggaaggagatgaaaaagagtacaatgctgatggaggaaagatgccaattaaatggatggctctggagtgtatacattacaggaaattcacccatcagagtgacgtttggagctatggagttactatatgggaactgatgacctttggaggaaaaccctatgatggaattccaacgcgagaaatccctgatttattagagaaaggagaacgtttgcctcagcctcccatctgcactattgacgtttacatggtcatggtcaaatgttggatgattgatgctgacagtagacctaaatttaaggaactggctgctgagttttcaaggatggctcgagaccctcaaagatacctagttattcagggtgatgatcgtatgaagcttcccagtccaaatgacagcaagttctttcagaatctcttggatgaagaggatttggaagatatgatggatgctgaggagtacttggtccctcaggctttcaacatcccacctcccatctatacttccagagcaagaattgactcgaataggagtgaaattggacacagccctcctcctgcctacacccccatgtcaggaaaccagtttgtataccgagatggaggttttgctgctgaacaaggagtgtctgtgccctacagagccccaactagcacaattccagaagctcctgtggcacagggtgctactgctgagatttttgatgactcctgctgtaatggcaccctacgcaagccagtggcaccccatgtccaagaggacagtagcacccagaggtacagtgctgaccccaccgtgtttgccccagaacggagcccacgaggagagctggatgaggaaggttacatgactcctatgcgagacaaacccaaacaagaatacctgaatccagtggaggagaacccttttgtttctcggagaaaaaatggagaccttcaagcattggataatcccgaatatcacaatgcatccaatggtccacccaaggccgaggatgagtatgtgaatgagccactgtacctcaacacctttgccaacaccttgggaaaagctgagtacctgaagaacaacatactgtcaatgccagagaaggccaagaaagcgtttgacaaccctgactactggaaccacagcctgccacctcggagcacccttcagcacccagactacctgcaggagtacagcacaaaatatttttataaacagaatgggcggatccggcctattgtggcagagaatcctgaatacctctctgagttctccctgaagccaggcactgtgctgccgcctccaccttacagacaccggaatactgtggtgtaagctcagttgtggttttttaggtggagagacacacctgctccaatttccccacccccctctctttctctggtggtcttccttctaccccaaggccagtagttttgacacttcccagtggaagatacagagatgcaatgatagttatgtgcttacctaacttgaacattagagggaaagactgaaagagaaagataggaggaaccacaatgtttcttcatttctctgcatgggttggtcaggagaatgaaacagctagagaaggaccagaaaatgtaaggcaatgctgcctactatcaaactagctgtcactttttttctttttctttttctttctttgtttctttcttcctcttctttttttttttttttttaaagcagatggttgaaacacccatgctatctgttcctatctgcaggaactgatgtgtgcatatttagcatccctggaaatcataataaagtttccattagaacaaaagaataacattttctataacatatgatagtgtctgaaattgagaatccagtttctttccccagcagtttctgtcctagcaagtaagaatggccaactcaactttcataatttaaaaatctccattaaagttataactagtaattatgttttcaacactttttggtttttttcattttgttttgctctgaccgattcctttatatttgctcccctatttttggctttaatttctaattgcaaagatgtttacatcaaagcttcttcacagaatttaagcaagaaatattttaatatagtgaaatggccactactttaagtatacaatctttaaaataagaaagggaggctaatatttttcatgctatcaaattatcttcaccctcatcctttacatttttcaacatttttttttctccataaatgacactacttgataggccgttggttgtctgaagagtagaagggaaactaagagacagttctctgtggttcaggaaaactactgatactttcaggggtggcccaatgagggaatccattgaactggaagaaacacactggattgggtatgtctacctggcagatactcagaaatgtagtttgcacttaagctgtaattttatttgttctttttctgaactccattttggattttgaatcaagcaatatggaagcaaccagcaaattaactaatttaagtacatttttaaaaaaagagctaagataaagactgtggaaatgccaaaccaagcaaattaggaaccttgcaacggtatccagggactatgatgagaggccagcacattatcttcatatgtcacctttgctacgcaaggaaatttgttcagttcgtatacttcgtaagaaggaatgcgagtaaggattggcttgaattccatggaatttctagtatgagactatttatatgaagtagaaggtaactctttgcacataaattggtataataaaaagaaaaacacaaacattcaaagcttagggataggtccttgggtcaaaagttgtaaataaatgtgaaacatcttctc

SEQ ID NO: 16 - protein, 1308 aa, Homo sapiens
MKPATGLWVWVSLLVAAGTVQPSDSQSVCAGTENKLSSLSDLEQQYRALRKYYENCEVVMGNLEITSIEHNRDLSFLRSVREVTGYVLVALNQFRYLPLENLRIIRGTKLYEDRYALAIFLNYRKDGNFGLQELGLKNLTEILNGGVYVDQNKFLCYADTIHWQDIVRNPWPSNLTLVSTNGSSGCGRCHKSCTGRCWGPTENHCQTLTRTVCAEQCDGRCYGPYVSDCCHRECAGGCSGPKDTDCFACMNFNDSGACVTQCPQTFVYNPTTFQLEHNFNAKYTYGAFCVKKCPHNFVVDSSSCVRACPSSKMEVEENGIKMCKPCTDICPKACDGIGTGSLMSAQTVDSSNIDKFINCTKINGNLIFLVTGIHGDPYNAIEAIDPEKLNVFRTVREITGFLNIQSWPPNMTDFSVFSNLVTIGGRVLYSGLSLLILKQQGITSLQFQSLKEISAGNIYITDNSNLCYYHTINWTTLFSTINQRIVIRDNRKAENCTAEGMVCNHLCSSDGCWGPGPDQCLSCRRFSRGRICIESCNLYDGEFREFENGSICVECDPQCEKMEDGLLTCHGPGPDNCTKCSHFKDGPNCVEKCPDGLQGANSFIFKYADPDRECHPCHPNCTQGCNGPTSHDCIYYPWTGHSTLPQHARTPLIAAGVIGGLFILVIVGLTFAVYVRRKSIKKKRALRRFLETELVEPLTPSGTAPNQAQLRILKETELKRVKVLGSGAFGTVYKGIWVPEGETVKIPVAIKILNETTGPKANVEFMDEALIMASMDHPHLVRLLGVCLSPTIQLVTQLMPHGCLLEYVHEHKDNIGSQLLLNWCVQIAKGMMYLEERRLVHRDLAARNVLVKSPNHVKITDFGLARLLEGDEKEYNADGGKMPIKWMALECIHYRKFTHQSDVWSYGVTIWELMTFGGKPYDGIPTREIPDLLEKGERLPQPPICTIDVYMVMVKCWMIDADSRPKFKELAAEFSRMARDPQRYLVIQGDDRMKLPSPNDSKFFQNLLDEEDLEDMMDAEEYLVPQAFNIPPPIYTSRARIDSNRSEIGHSPPPAYTPMSGNQFVYRDGGFAAEQGVSVPYRAPTSTIPEAPVAQGATAEIFDDSCCNGTLRKPVAPHVQEDSSTQRYSADPTVFAPERSPRGELDEEGYMTPMRDKPKQEYLNPVEENPFVSRRKNGDLQALDNPEYHNASNGPPKAEDEYVNEPLYLNTFANTLGKAEYLKNNILSMPEKAKKAFDNPDYWNHSLPPRSTLQHPDYLQEYSTKYFYKQNGRIRPIVAENPEYLSEFSLKPGTVLPPPPYRHRNTVV

SEQ ID NO: 17 - DNA, 4989 bp, Homo sapiens
  CDS (46)..(4149): IGF-1R coding sequence
ttttttttttttttgagaaagggaatttcatcccaaataaaaggaatgaagtctggctccggaggagggtccccgacctcgctgtgggggctcctgtttctctccgccgcgctctcgctctggccgacgagtggagaaatctgcgggccaggcatcgacatccgcaacgactatcagcagctgaagcgcctggagaactgcacggtgatcgagggctacctccacatcctgctcatctccaaggccgaggactaccgcagctaccgcttccccaagctcacggtcattaccgagtacttgctgctgttccgagtggctggcctcgagagcctcggagacctcttccccaacctcacggtcatccgcggctggaaactcttctacaactacgccctggtcatcttcgagatgaccaatctcaaggatattgggctttacaacctgaggaacattactcggggggccatcaggattgagaaaaatgctgacctctgttacctctccactgtggactggtccctgatcctggatgcggtgtccaataactacattgtggggaataagcccccaaaggaatgtggggacctgtgtccagggaccatggaggagaagccgatgtgtgagaagaccaccatcaacaatgagtacaactaccgctgctggaccacaaaccgctgccagaaaatgtgcccaagcacgtgtgggaagcgggcgtgcaccgagaacaatgagtgctgccaccccgagtgcctgggcagctgcagcgcgcctgacaacgacacggcctgtgtagcttgccgccactactactatgccggtgtctgtgtgcctgcctgcccgcccaacacctacaggtttgagggctggcgctgtgtggaccgtgacttctgcgccaacatcctcagcgccgagagcagcgactccgaggggtttgtgatccacgacggcgagtgcatgcaggagtgcccctcgggcttcatccgcaacggcagccagagcatgtactgcatcccttgtgaaggtccttgcccgaaggtctgtgaggaagaaaagaaaacaaagaccattgattctgttacttctgctcagatgctccaaggatgcaccatcttcaagggcaatttgctcattaacatccgacgggggaataacattgcttcagagctggagaacttcatggggctcatcgaggtggtgacgggctacgtgaagatccgccattctcatgccttggtctccttgtccttcctaaaaaaccttcgcctcatcctaggagaggagcagctagaagggaattactccttctacgtcctcgacaaccagaacttgcagcaactgtgggactgggaccaccgcaacctgaccatcaaagcagggaaaatgtactttgctttcaatcccaaattatgtgtttccgaaatttaccgcatggaggaagtgacggggactaaagggcgccaaagcaaaggggacataaacaccaggaacaacggggagagagcctcctgtgaaagtgacgtcctgcatttcacctccaccaccacgtcgaagaatcgcatcatcataacctggcaccggtaccggccccctgactacagggatctcatcagcttcaccgtttactacaaggaagcaccctttaagaatgtcacagagtatgatgggcaggatgcctgcggctccaacagctggaacatggtggacgtggacctcccgcccaacaaggacgtggagcccggcatcttactacatgggctgaagccctggactcagtacgccgtttacgtcaaggctgtgaccctcaccatggtggagaacgaccatatccgtggggccaagagtgagatcttgtacattcgcaccaatgcttcagttccttccattcccttggacgttctttcagcatcgaactcctcttctcagttaatcgtgaagtggaaccctccctctctgcccaacggcaacctgagttactacattgtgcgctggcagcggcagcctcaggacggctacctttaccggcacaattactgctccaaagacaaaatccccatcaggaagtatgccgacggcaccatcgacattgaggaggtcacagagaaccccaagactgaggtgtgtggtggggagaaagggccttgctgcgcctgccccaaaactgaagccgagaagcaggccgagaaggaggaggctgaataccgcaaagtctttgagaatttcctgcacaactccatcttcgtgcccagacctgaaaggaagcggagagatgtcatgcaagtggccaacaccaccatgtccagccgaagcaggaacaccacggccgcagacacctacaacatcaccgacccggaagagctggagacagagtaccctttctttgagagcagagtggataacaaggagagaactgtcatttctaaccttcggcctttcacattgtaccgcatcgatatccacagctgcaaccacgaggctgagaagctgggctgcagcgcctccaacttcgtctttgcaaggactatgcccgcagaaggagcagatgacattcctgggccagtgacctgggagccaaggcctgaaaactccatctttttaaagtggccggaacctgagaatcccaatggattgattctaatgtatgaaataaaatacggatcacaagttgaggatcagcgagaatgtgtgtccagacaggaatacaggaagtatggaggggccaagctaaaccggctaaacccggggaactacacagcccggattcaggccacatctctctctgggaatgggtcgtggacagatcctgtgttcttctatgtccaggccaaaacaggatatgaaaacttcatccatctgatcatcgctctgcccgtcgctgtcctgttgatcgtgggagggttggtgattatgctgtacgtcttccatagaaagagaaataacagcaggctggggaatggagtgctgtatgcctctgtgaacccggagtacttcagcgctgctgatgtgtacgttcctgatgagtgggaggtggctcgggagaagatcaccatgagccgggaacttgggcaggggtcgtttgggatggtctatgaaggagttgccaagggtgtggtgaaagatgaacctgaaaccagagtggccattaaaacagtgaacgaggccgcaagcatgcgtgagaggattgagtttctcaacgaagcttctgtgatgaaggagttcaattgtcaccatgtggtgcgattgctgggtgtggtgtcccaaggccagccaacactggtcatcatggaactgatgacacggggcgatctcaaaagttatctccggtctctgaggccagaaatggagaataatccagtcctagcacctccaagcctgagcaagatgattcagatggccggagagattgcagacggcatggcatacctcaacgccaataagttcgtccacagagaccttgctgcccggaattgcatggtagccgaagatttcacagtcaaaatcggagattttggtatgacgcgagatatctatgagacagactattaccggaaaggaggcaaagggctgctgcccgtgcgctggatgtctcctgagtccctcaaggatggagtcttcaccacttactcggacgtctggtccttcggggtcgtcctctgggagatcgccacactggccgagcagccctaccagggcttgtccaacgagcaagtccttcgcttcgtcatggagggcggccttctggacaagccagacaactgtcctgacatgctgtttgaactgatgcgcatgtgctggcagtataaccccaagatgaggccttccttcctggagatcatcagcagcatcaaagaggagatggagcctggcttccgggaggtctccttctactacagcgaggagaacaagctgcccgagccggaggagctggacctggagccagagaacatggagagcgtccccctggacccctcggcctcctcgtcctccctgccactgcccgacagacactcaggacacaaggccgagaacggccccggccctggggtgctggtcctccgcgccagcttcgacgagagacagccttacgcccacatgaacgggggccgcaagaacgagcgggccttgccgctgccccagtcttcgacctgctgatccttggatcctgaatctgtgcaaacagtaacgtgtgcgcacgcgcagcggggtggggggggagagagagttttaacaatccattcacaagcctcctgtacctcagtggatcttcagttctgcccttgctgcccgcgggagacagcttctctgcagtaaaacacatttgggatgttccttttttcaatatgcaagcagctttttattccctgcccaaacccttaactgacatgggcctttaagaaccttaatgacaacacttaatagcaacagagcacttgagaaccagtctcctcactctgtccctgtccttccctgttctccctttctctctcctctctgcttcataacggaaaaataattgccacaagtccagctgggaagccctttttatcagtttgaggaagtggctgtccctgtggccccatccaaccactgtacacacccgcctgacaccgtgggtcattacaaaaaaacacgtggagatggaaatttttacctttatctttcacctttctagggacatgaaatttacaaagggccatcgttcatccaaggctgttaccattttaacgctgcctaattttgccaaaatcctgaactttctccctcatcggcccggcgctgattcctcgtgtccggaggcatgggtgagcatggcagctggttgctccatttgagagacacgctggcgacacactccgtccatccgactgcccctgctgtgctgctcaaggccacaggcacacaggtctcattgcttctgactagattattatttgggggaactggacacaataggtctttctctcagtgaaggtggggagaagctgaaccggc

SEQ ID NO: 18 - protein, 1367 aa, Homo sapiens
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 19 - DNA, 4691 bp, Homo sapiens
  CDS (107)..(4255): coding sequence
gagaaggacgcgcggcccccagcgctcttgggggccgcctcggagcatgacccccgcgggccagcgccgcgcgcctgatccgaggagaccccgcgctcccgcagccatgggcaccgggggccggcggggggcggcggccgcgccgctgctggtggcggtggccgcgctgctactgggcgccgcgggccacctgtaccccggagaggtgtgtcccggcatggatatccggaacaacctcactaggttgcatgagctggagaattgctctgtcatcgaaggacacttgcagatactcttgatgttcaaaacgaggcccgaagatttccgagacctcagtttccccaaactcatcatgatcactgattacttgctgctcttccgggtctatgggctcgagagcctgaaggacctgttccccaacctcacggtcatccggggatcacgactgttctttaactacgcgctggtcatcttcgagatggttcacctcaaggaactcggcctctacaacctgatgaacatcacccggggttctgtccgcatcgagaagaacaatgagctctgttacttggccactatcgactggtcccgtatcctggattccgtggaggataatcacatcgtgttgaacaaagatgacaacgaggagtgtggagacatctgtccgggtaccgcgaagggcaagaccaactgccccgccaccgtcatcaacgggcagtttgtcgaacgatgttggactcatagtcactgccagaaagtttgcccgaccatctgtaagtcacacggctgcaccgccgaaggcctctgttgccacagcgagtgcctgggcaactgttctcagcccgacgaccccaccaagtgcgtggcctgccgcaacttctacctggacggcaggtgtgtggagacctgcccgcccccgtactaccacttccaggactggcgctgtgtgaacttcagcttctgccaggacctgcaccacaaatgcaagaactcgcggaggcagggctgccaccaatacgtcattcacaacaacaagtgcatccctgagtgtccctccgggtacacgatgaattccagcaacttgctgtgcaccccatgcctgggtccctgtcccaaggtgtgccacctcctagaaggcgagaagaccatcgactcggtgacgtctgcccaggagctccgaggatgcaccgtcatcaacgggagtctgatcatcaacattcgaggaggcaacaatctggcagctgagctagaagccaacctcggcctcattgaagaaatttcagggtatctaaaaatccgccgatcctacgctctggtgtcactttccttcttccggaagttacgtctgattcgaggagagaccttggaaattgggaactactccttctatgccttggacaaccagaacctaaggcagctctgggactggagcaaacacaacctcaccaccactcaggggaaactcttcttccactataaccccaaactctgcttgtcagaaatccacaagatggaagaagtttcaggaaccaaggggcgccaggagagaaacgacattgccctgaagaccaatggggacaaggcatcctgtgaaaatgagttacttaaattttcttacattcggacatcttttgacaagatcttgctgagatgggagccgtactggccccccgacttccgagacctcttggggttcatgctgttctacaaagaggccccttatcagaatgtgacggagttcgatgggcaggatgcgtgtggttccaacagttggacggtggtagacattgacccacccctgaggtccaacgaccccaaatcacagaaccacccagggtggctgatgcggggtctcaagccctggacccagtatgccatctttgtgaagaccctggtcaccttttcggatgaacgccggacctatggggccaagagtgacatcatttatgtccagacagatgccaccaacccctctgtgcccctggatccaatctcagtgtctaactcatcatcccagattattctgaagtggaaaccaccctccgaccccaatggcaacatcacccactacctggttttctgggagaggcaggcggaagacagtgagctgttcgagctggattattgcctcaaagggctgaagctgccctcgaggacctggtctccaccattcgagtctgaagattctcagaagcacaaccagagtgagtatgaggattcggccggcgaatgctgctcctgtccaaagacagactctcagatcctgaaggagctggaggagtcctcgtttaggaagacgtttgaggattacctgcacaacgtggttttcgtccccagaaaaacctcttcaggcactggtgccgaggaccctaggccatctcggaaacgcaggtcccttggcgatgttgggaatgtgacggtggccgtgcccacggtggcagctttccccaacacttcctcgaccagcgtgcccacgagtccggaggagcacaggccttttgagaaggtggtgaacaaggagtcgctggtcatctccggcttgcgacacttcacgggctatcgcatcgagctgcaggcttgcaaccaggacacccctgaggaacggtgcagtgtggcagcctacgtcagtgcgaggaccatgcctgaagccaaggctgatgacattgttggccctgtgacgcatgaaatctttgagaacaacgtcgtccacttgatgtggcaggagccgaaggagcccaatggtctgatcgtgctgtatgaagtgagttatcggcgatatggtgatgaggagctgcatctctgcgtctcccgcaagcacttcgctctggaacggggctgcaggctgcgtgggctgtcaccggggaactacagcgtgcgaatccgggccacctcccttgcgggcaacggctcttggacggaacccacctatttctacgtgacagactatttagacgtcccgtcaaatattgcaaaaattatcatcggccccctcatctttgtctttctcttcagtgttgtgattggaagtatttatctattcctgagaaagaggcagccagatgggccgctgggaccgctttacgcttcttcaaaccctgagtatctcagtgccagtgatgtgtttccatgctctgtgtacgtgccggacgagtgggaggtgtctcgagagaagatcaccctccttcgagagctggggcagggctccttcggcatggtgtatgagggcaatgccagggacatcatcaagggtgaggcagagacccgcgtggcggtgaagacggtcaacgagtcagccagtctccgagagcggattgagttcctcaatgaggcctcggtcatgaagggcttcacctgccatcacgtggtgcgcctcctgggagtggtgtccaagggccagcccacgctggtggtgatggagctgatggctcacggagacctgaagagctacctccgttctctgcggccagaggctgagaataatcctggccgccctccccctacccttcaagagatgattcagatggcggcagagattgctgacgggatggcctacctgaacgccaagaagtttgtgcatcgggacctggcagcgagaaactgcatggtcgcccatgattttactgtcaaaattggagactttggaatgaccagagacatctatgaaacggattactaccggaaagggggcaagggtctgctccctgtacggtggatggcaccggagtccctgaaggatggggtcttcaccacttcttctgacatgtggtcctttggcgtggtcctttgggaaatcaccagcttggcagaacagccttaccaaggcctgtctaatgaacaggtgttgaaatttgtcatggatggagggtatctggatcaacccgacaactgtccagagagagtcactgacctcatgcgcatgtgctggcaattcaaccccaagatgaggccaaccttcctggagattgtcaacctgctcaaggacgacctgcaccccagctttccagaggtgtcgttcttccacagcgaggagaacaaggctcccgagagtgaggagctggagatggagtttgaggacatggagaatgtgcccctggaccgttcctcgcactgtcagagggaggaggcggggggccgggatggagggtcctcgctgggtttcaagcggagctacgaggaacacatcccttacacacacatgaacggaggcaagaaaaacgggcggattctgaccttgcctcggtccaatccttcctaacagtgcctaccgtggcgggggcgggcaggggttcccattttcgctttcctctggtttgaaagcctctggaaaactcaggattctcacgactctaccatgtccagtggagttcagagatcgttcctatacatttctgttcatcttaaggtggactcgtttggttaccaatttaactagtcctgcagaggatttaactgtgaacctggagggcaaggggtttccacagttgctgctcctttggggcaacgacggtttcaaaccaggattttgtgttttttcgttccccccacccgcccccagcagatggaaagaaagcacctgtttttacaaattcttttttttttttttttttttttttttttgctggtgtctgagcttcagtataaaagacaaaacttcctgtttgtggaacaaaatttcgaaagaaaaaaccaaa

SEQ ID NO: 20 - protein, 1382 aa, Homo sapiens
MGTGGRRGAAAAPLLVAVAALLLGAAGHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNHIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTTTQGKLFFHYNPKLCLSEIHKMEEVSGTKGRQERNDIALKTNGDKASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRKTSSGTGAEDPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAKIIIGPLIFVFLFSVVIGSIYLFLRKRQPDGPLGPLYASSNPEYLSASDVFPCSVYVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENKAPESEELEMEFEDMENVPLDRSSHCQREEAGGRDGGSSLGFKRSYEEHIPYTHMNGGKKNGRILTLPRSNPS

SEQ ID NO: 21 - protein, 79 aa, Homo sapiens
GTHSLPPRPAAVPVPLRMQPGPAHPVLSFLRPSWDLVSAFYSLPLAPLSPTSVPISPVSVGRGPDPDAHVAVDLSRYEG

SEQ ID NO: 22 - protein, 79 aa, Homo sapiens
  MISC_FEATURE (2)..(2): Xaa reflects Thr or Ser variants
GXHSLPPRPAAVPVPLRMQPGPAHPVLSFLRPSWDLVSAFYSLPLAPLSPTSVPISPVSVGRGPDPDAHVAVDLSRYEG

SEQ ID NO: 23 - protein, 79 aa, Homo sapiens
  MISC_FEATURE (5)..(5): Xaa reflects Leu or Pro variants
GTHSXPPRPAAVPVPLRMQPGPAHPVLSFLRPSWDLVSAFYSLPLAPLSPTSVPISPVSVGRGPDPDAHVAVDLSRYEG

SEQ ID NO: 24 - protein, 79 aa, Homo sapiens
  MISC_FEATURE (6)..(6): Xaa reflects Pro or Leu variants
GTHSLXPRPAAVPVPLRMQPGPAHPVLSFLRPSWDLVSAFYSLPLAPLSPTSVPISPVSVGRGPDPDAHVAVDLSRYEG

SEQ ID NO: 25 - protein, 79 aa, Homo sapiens
  MISC_FEATURE (16)..(16): Xaa reflects Leu or Gln variants
GTHSLPPRPAAVPVPXRMQPGPAHPVLSFLRPSWDLVSAFYSLPLAPLSPTSVPISPVSVGRGPDPDAHVAVDLSRYEG

SEQ ID NO: 26 - protein, 79 aa, Homo sapiens
  MISC_FEATURE (17)..(17): Xaa reflects Arg or Cys variants
GTHSLPPRPAAVPVPLRXQPGPAHPVLSFLRPSWDLVSAFYSLPLAPLSPTSVPISPVSVGRGPDPDAHVAVDLSRYEG

SEQ ID NO: 27 - protein, 79 aa, Homo sapiens
  MISC_FEATURE (18)..(18): Xaa reflects Met or Leu variants
GTHSLPPRPAAVPVPLRMQPXPAHPVLSFLRPSWDLVSAFYSLPLAPLSPTSVPISPVSVGRGPDPDAHVAVDLSRYEG

SEQ ID NO: 28 - protein, 79 aa, Homo sapiens
  MISC_FEATURE (21)..(21): Xaa reflects Gly, Asp, Ala or Val variants
GTHSLPPRPAAVPVPLRMQPGPAHPVLSFLRPSWDXVSAFYSLPLAPLSPTSVPISPVSVGRGPDPDAHVAVDLSRYEG

SEQ ID NO: 29 - protein, 79 aa, Homo sapiens
  MISC_FEATURE (31)..(31): Xaa reflects Arg or Ile variants
GTHSLPPRPAAVPVPLRMQPGPAHPVLSFLRPSWDLVSAFYSLPLAPLSPTSVXISPVSVGRGPDPDAHVAVDLSRYEG

SEQ ID NO: 30 - protein, 79 aa, Homo sapiens
  MISC_FEATURE (36)..(36): Xaa reflects Leu or Ile variants
GTHSLPPRPAAVPVPLRMQPGPAHPVLSFLRPSWDLVSAFYSLPLAPLSPTSVPISPVSVGRGXDPDAHVAVDLSRYEG

SEQ ID NO: 31 - protein, 79 aa, Homo sapiens
  MISC_FEATURE (54)..(54): Xaa reflects Pro or Arg variants
GTHSLPPRPAAVPVPLRMQPGPAHPVLSFLRPSWDLVSAFYSLPLAPLSPTSVPISPVSVGRGPDPDAHVAVXLSRYEG

SEQ ID NO: 32 - protein, 419 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARGTHSLPPRPAAVPVPLRMQPGPAHPVLSFLRPSWDLVSAFYSLPLAPLSPTSVPISPVSVGRGPDPDAHVAVDLSRYEG

SEQ ID NO: 33 - protein, 419 aa, Homo sapiens
  MISC_FEATURE (342)..(342): Xaa reflects Thr or Ser variants
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARGXHSLPPRPAAVPVPLRMQPGPAHPVLSFLRPSWDLVSAFYSLPLAPLSPTSVPISPVSVGRGPDPDAHVAVDLSRYEG

SEQ ID NO: 34 - protein, 419 aa, Homo sapiens
  MISC_FEATURE (345)..(345): Xaa reflects Leu or Pro variants
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARGTHSXPPRPAAVPVPLRMQPGPAHPVLSFLRPSWDLVSAFYSLPLAPLSPTSVPISPVSVGRGPDPDAHVAVDLSRYEG

SEQ ID NO: 35 - protein, 419 aa, Homo sapiens
  MISC_FEATURE (346)..(346): Xaa reflects Pro or Leu variants
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARGTHSLXPRPAAVPVPLRMQPGPAHPVLSFLRPSWDLVSAFYSLPLAPLSPTSVPISPVSVGRGPDPDAHVAVDLSRYEG

SEQ ID NO: 36 - protein, 419 aa, Homo sapiens
  MISC_FEATURE (356)..(356): Xaa reflects Leu or Gln variants
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARGTHSLPPRPAAVPVPXRMQPGPAHPVLSFLRPSWDLVSAFYSLPLAPLSPTSVPISPVSVGRGPDPDAHVAVDLSRYEG

SEQ ID NO: 37 - protein, 419 aa, Homo sapiens
  MISC_FEATURE (357)..(357): Xaa reflects Arg or Cys variants
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARGTHSLPPRPAAVPVPLRXQPGPAHPVLSFLRPSWDLVSAFYSLPLAPLSPTSVPISPVSVGRGPDPDAHVAVDLSRYEG

SEQ ID NO: 38 - protein, 419 aa, Homo sapiens
  MISC_FEATURE (358)..(358): Xaa reflects Met or Leu variants
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARGTHSLPPRPAAVPVPLRMQPXPAHPVLSFLRPSWDLVSAFYSLPLAPLSPTSVPISPVSVGRGPDPDAHVAVDLSRYEG

SEQ ID NO: 39 - protein, 419 aa, Homo sapiens
  MISC_FEATURE (361)..(361): Xaa reflects Gly, Asp, Ala or Val variants
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARGTHSLPPRPAAVPVPLRMQPGPAHPVLSFLRPSWDXVSAFYSLPLAPLSPTSVPISPVSVGRGPDPDAHVAVDLSRYEG

SEQ ID NO: 40 - protein, 419 aa, Homo sapiens
  MISC_FEATURE (371)..(371): Xaa reflects Arg or Ile variants
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARGTHSLPPRPAAVPVPLRMQPGPAHPVLSFLRPSWDLVSAFYSLPLAPLSPTSVXISPVSVGRGPDPDAHVAVDLSRYEG

SEQ ID NO: 41 - protein, 419 aa, Homo sapiens
  MISC_FEATURE (376)..(376): Xaa reflects Leu or Ile variants
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARGTHSLPPRPAAVPVPLRMQPGPAHPVLSFLRPSWDLVSAFYSLPLAPLSPTSVPISPVSVGRGXDPDAHVAVDLSRYEG

SEQ ID NO: 42 - protein, 419 aa, Homo sapiens
  MISC_FEATURE (394)..(394): Xaa reflects Pro or Arg variants
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARGTHSLPPRPAAVPVPLRMQPGPAHPVLSFLRPSWDLVSAFYSLPLAPLSPTSVPISPVSVGRGPDPDAHVAVXLSRYEG

(19) United States
(12) Patent Application Publication
(10) Pub. No.: US 2008/0206231 A1
(43) Pub. Date: Aug. 28, 2008

(54) Compositions and Methods for Treating Disease

(75) Inventors: Gail M. Clinton, Wimberley, TX (US); Charles T. Roberts, Portland, OR (US)

Correspondence Address: DAVIS WRIGHT TREMAINE, LLP/Seattle, 1201 Third Avenue, Suite 2200, SEATTLE, WA, 98101-3045, US

(73) Assignee: OREGON HEALTH AND SCIENCE UNIVERSITY, Portland, OR (US)

(21) Appl. No.: 11/576,996

(22) PCT Filed: Oct. 5, 2005
(86) PCT No.: PCT/US05/35961

§ 371 Date: Jan. 28, 2008

Related U.S. Application Data
(60) Provisional application No. 60/616,596, filed on Oct. 5, 2004.
(60) Provisional application No. 60/688,355, filed on Jun. 6, 2005.

(51) Int. Cl. A61K 39/395; A61K 38/28; A61K 38/17; C12N 5/00

(52) U.S. Cl. 424/130.1; 514/12; 514/4; 435/325

(57) ABSTRACT

The present invention discloses for the first time that the insulin receptor (IR) is a target of Herstatin, which modulates IR and IR-mediated intracellular signaling. In preferred aspects, Herstatin binds at nM concentrations to cell-surface IR, up-regulates basal IR expression by several-fold, induces the accumulation of pro-IR, and stimulates insulin activation of the ERK pathway. Moreover, these changes in insulin signaling are accompanied by alterations in IGF-IR expression, IRS-2 levels, and the serine phosphorylation state of both IRS-1 and IRS-2. Preferred aspects provide novel therapeutic methods and pharmaceutical compositions for treatment of conditions associated with altered IR expression or IR-mediated signaling, including but not limited to insulin resistance syndrome, pre-diabetic conditions, metabolic syndrome, type 1 and type 2 diabetes, cardiac disease, diabetes-associated vascular disease, atherosclerosis, hypertension, diabetes-associated lipid metabolism disorders (dyslipidemia), obesity, critical illness, neurodegenerative disorders, and combinations thereof, and cancer.

### CROSS-REFERENCE TO RELATED APPLICATIONS

This application claims the benefit of priority to: U.S. Provisional Patent Application Ser. No. 60/616,596, filed 5 Oct. 2004 and entitled “COMPOSITIONS AND METHODS FOR TREATING DISEASE”; and to U.S. Provisional Patent Application Ser. No. 60/688,355, filed 6 Jun. 2005, of same title, both of which are incorporated by reference herein in their entireties.

### FIELD OF THE INVENTION

Aspects of the invention relate generally to therapeutic molecules, compositions and methods for treatment of diseases through modulation of the insulin receptor (IR) and IR-mediated intracellular signaling by administration of Herstatin or variants thereof, and in more particular aspects relate to compositions and methods for cell targeting, and for the treatment of conditions or diseases associated with altered IR expression or altered IR-mediated signaling, including but not limited to insulin resistance syndrome, pre-diabetic conditions, metabolic syndrome, type 1 and type 2 diabetes, cardiac disease, diabetes-associated vascular disease, atherosclerosis, hypertension, diabetes-associated lipid metabolism disorders (dyslipidemia), obesity, critical illness, neurodegenerative disorders, and combinations thereof, and cancer.

### BACKGROUND

The Insulin Receptor. The insulin receptor is the canonical member of the insulin receptor family of receptor tyrosine kinases, which also includes the IGF-IR and the insulin receptor-related receptor (IRR). These molecules share a heterotetrameric structure comprised of two extracellular ligand-binding α subunits, which are coupled to each other and to two transmembrane β subunits by disulfide linkages. The intracellular portion of the β subunit contains the intrinsic tyrosine kinase catalytic domain, which is activated by binding of extracellular ligand and a presumed conformational change in the β subunit. The activated receptor undergoes autophosphorylation of tyrosine residues in the kinase domain as well as residues in the flanking juxtamembrane and carboxyl-terminal domains. The phosphorylation of these residues, particularly in the juxtamembrane region, allows the recruitment of scaffolding adapter proteins such as IRS-1 and IRS-2 and Shc, which are then phosphorylated on tyrosine residues by the activated receptor to recruit a second level of signaling molecules to initiate the signaling cascades that are responsible for insulin action. These include the ERK arm of the MAPK pathway, the P13K-Akt/PKB pathway, and the APS-Cbl-CrkII-TC10 pathway. In cells expressing both insulin and IGF-I receptors, hybrid receptors consisting of insulin and IGF-I receptor α-β hemireceptors can form. These are activated by IGF-I but not by insulin. The insulin receptor family of receptors differs from the erbB/Her receptors by virtue of their existence as pre-dimerized heterotetramers and their use of intermediates such as IRS and Shc proteins to couple to downstream signaling pathways.

Diabetes and Related Conditions. The epidemic of obesity occurring in the in the United States and around the world portends a significant increase in type 2 diabetes mellitus in the adult and, increasingly, in the pediatric populations. There is also growing concern regarding the prevalence of pre-diabetic conditions such as the metabolic syndrome, the incidence of which dwarfs that of clinically apparent diabetes per se. The hyperglycemia of type 2 diabetes results from defects in both insulin sensitivity and pancreatic β-cell function, leading to a relatively insulin-deficient state. There is also a growing appreciation that insulin resistance may play an important role in cardiac disease. A mainstay of current therapy is the use of insulin-sensitizing agents such as metformin and thiazolidinediones that act to enhance the ability of insulin to trigger appropriate cellular responses such as glucose transport in insulin target tissues. These treatments suffer, however, from a lack of mechanistic specificity, high. rates of unresponsiveness (up to 30% for thiazolidinediones), and frequent side effects. Although advances are being made in the generation of islets for transplant, the time frame for the successful application of these approaches in human patients with both type 1 and type 2 disease and their ability to affect insulin resistance remains unclear. Thus, there continues to be an urgent need to design new and novel therapies to treat insulin resistance (see, e.g., Alsheikh-Ali & Karas, Amer J Cardiology, 93:1417-8, 2004; Ovalle & Fernando, Southern Med J., 95:1188-94, 2002; and Zangeneh et al., Mayo Clinic Proc. 78:471-479, 2003).).

The ErbB Receptor Family. The ErbB receptor family consists of four receptor tyrosine kinases: EGFR (HER-1, erbB-1), HER-2 (neu, erbB-2), HER-3 (erbB-3) and HER-4 (erbB-4). Altered expression of ErbB receptors by mutational activation, receptor overexpression, and tumor production of ligands contributes to the development and maintenance of a variety of human cancers (Olayioye et al., Embo J., 19:3159-67, 2000).

The ErbB receptors are activated by several ligands with an EGF core domain (EGF-related growth factors). The exception is the HER-2 receptor, which is recruited as a preferred dimer partner with other ligand binding erbB receptors (Id.). The eleven mammalian EGF-like ligands are all agonists, whereas Drosophila express the ligand Argos that inhibits activation of the EGFR (Dougall et al., Oncogene 9:2109-23, 1994; Hynes & Stem, Biochim. Biophys. Acta 1198:165-84, 1994); Tzahar & Yarden, Biochim. Biophys. Acta 1377:25-37, 1998).

Insulin-like growth factor 1 receptor (IGF-IR). Anti-erbB receptor antibody agents, such as the HER-2-specific antibody rhuMAb4D5 (HERCEPTIN™) have been approved for cancer therapy. Significantly, however, tumor cells may be inherently resistant, or gain resistance, to anti-erbB receptor therapies through activation of IGF-IR pathways (Chakravarti et al., Cancer Res. 62:200-7, 2002; Lu et al., J. Biol. Chem. 279:2856-65, 2004; Lu et al., J. Natl. Cancer Inst., 93:1852-7, 2001). Activation of the IGF-IR by IGF-I promotes, inter alia, proliferation, survival, transformation, metastasis, and angiogenesis (Baserga, Hum. Pathol. 31, 275-6, 2000; Wang & Sun, Curr. Cancer Drug Targets 2:191-207, 2002), and signaling through both IGF-IR and EGF receptors is central to tumorigenesis. IGF-IR is in the same receptor family as the insulin receptor.

Herstatin. Although the HER-2 receptor does not directly bind EGF-like ligands, a secreted product of an HER-2 alternative transcript, Herstatin, binds with high affinity to the ectodomains of all members of the EGF receptor family, including EGFR/HER1/erbB1, HER2/neu/erbB2, HER3/erbB3, and HER4/erbB4, and to ΔEGFR and IGF-IR (Shamieh et al., FEBS Letters, 568:163-166, 2004). Herstatin was originally cloned from ovarian cancer cells, and consists of a segment (340 amino acids identical to the N-terminal subdomains I and II) of the HER-2 ectodomain, followed by 79 amino acids, encoded by intron 8 that function as a receptor binding domain (RBD) (Doherty et al., Proc. Natl. Acad. Sci. USA 96:10869-74, 1999). Herstatin blocks homomeric and heteromeric ErbB receptor interactions (e.g., dimerization and activation), inhibits signaling by EGR ligands and by IGF-1 (e.g., inhibits activation of the P13K/Akt pathway initiated by EGF, TGFα, Heregulin and IGF-1) (Doherty et al., Proc Natl Acad Sci., 96:10869-10874, 1999; Azios et al., Oncogene, 20:5199-5209, 2001; Justman & Clinton, J Biol Chem., 277:20618-20624, 2002; Jhabvala-Romero et al., Oncogene, 22:8178-8186, 2003; and Shamieh et al., supra), causes growth arrest, and has utility as an anti-cancer agent (Id., Azios et al., Oncogene 20:5199-209, 2001; Jhabvala-Romero et al., Oncogene 22:8178-86, 2003; Justman & Clinton, J. Biol. Chem. 277:20618-24, 2002).

There is, therefore, a need in the art to further investigate and characterize the interactions among the IR, the erbB family receptors, and the IGF-I receptor, and to identify modulators of the signaling mediated by these receptors.

There is a pronounced need in the art to identify and develop IR modulators as therapeutic agents.

There is a pronounced need in the art to design new and novel therapies to treat insulin resistance.

There is a need in the art to further assess and exploit the receptor-modulating utilities of Herstatin.

### SUMMARY OF THE INVENTION

The present invention relates to therapeutic molecules and compositions for modulation of the insulin receptor (IR) and IR-mediated intracellular signaling by administration of an isoform of a cell surface receptor, and in preferred aspects, to administration of Herstatin, which is an example of such a cell surface receptor isoform. Aspects of the invention are based upon the discovery that the insulin receptor (IR) is a target of Herstatin, which specifically binds to the IR with nM affinity. According to preferred aspects of the present invention, Herstatin alters the landscape of IR-mediated signaling, exerting a positive effect on IR expression, and substantially increasing IR-mediated ERK pathway activation. The MEK (MAPK kinase)-ERK pathway has been shown to be significantly involved in glucose transport (e.g., Harmon et al., Am. J. Physiol. Endocrinol. Metab., 287:E758-E766, 2004).

In particular aspects, Herstatin was shown herein to bind at nM concentrations to cell-surface IR, to up-regulate basal IR expression by several-fold, and to induce the accumulation of pro-IR.

In additional aspects, and with respect to signal transduction, Herstatin was shown herein to substantially (e.g., >40-fold) stimulate insulin activation of the ERK pathway, but to have little effect on insulin-stimulated activation of the P13K/Akt pathway.

In further aspects, these changes in insulin signaling were shown herein to be accompanied by about a 4-fold decrease in IGF-IR expression, a decrease in the apparent serine phosphorylation state of IRS-1, and a slight decrease in IRS-2 levels as well as a decrease in apparent serine phosphorylation of IRS-2.

Therefore, according to particular aspects of the present invention, Herstatin, a cell surface receptor isoform, has substantial utility for modulating insulin signaling in cells expressing IR.

Preferred aspects of the present invention thus provide novel therapeutic methods and pharmaceutical compositions comprising a cell surface receptor isoform (e.g., Herstatin, and/or variants thereof) for modulating IR, and IR-mediated signal transduction.

Alternative preferred aspects provide for a novel use of Herstatin in therapeutic methods and pharmaceutical compositions for treating various diseases associated with or characterized by alterations in insulin sensitivity or resistance (e.g., conditions or diseases characterized by altered IR expression and/or altered IR-related signaling).

In preferred embodiments, the invention provides novel methods and compositions for the treatment of conditions or diseases associated with altered IR expression or altered IR-mediated signaling, including but not limited to at least one of insulin resistance syndrome, pre-diabetic conditions, metabolic syndrome, type 1 and type 2 diabetes, cardiac disease, diabetes-associated vascular disease, atherosclerosis, hypertension, diabetes-associated lipid metabolism disorders (dyslipidemia), obesity, critical illness, neurodegenerative disorders, and cancer.

Additional aspects provide novel methods of targeted drug delivery.

Methods of treatment. Particularly preferred embodiments provide a method for treating or modulating a condition having an aspect related to, or associated with, or characterized by altered IR expression or altered IR-mediated signaling at a cellular level, comprising administering to a subject having such a condition, a therapeutically effective amount of a cell surface receptor isoform such as Herstatin, or a variant thereof (e.g., a therapeutically effective amount of a Int8 RBD polypeptide, or a variant thereof), that binds to the extracellular domain of cellular target IR. Preferably, the condition is selected from the group consisting of insulin resistance, pre-diabetic conditions, metabolic syndrome, type 1 and type 2 diabetes, cardiac disease, diabetes-associated vascular disease, diabetes-associated lipid metabolism disorders, neurodegenerative disorders, and combinations thereof. In alternative related embodiments, the cell further expresses a target receptor selected from the group consisting of: EGFR (HER-1, erbB-1); ΔEGFR; HER-2 (erbB-2); HER-3 (erbB-3); HER-4 (erbB-4); IGF-IR and combinations thereof.

Alternative related preferred embodiments further comprise administering a therapeutically effective amount of a molecule such as a small molecule, protein, peptide or receptor-specific antibody that binds to the extracellular domain of a target receptor selected from the group consisting of: IR, EGFR (HER-1, erbB-1); ΔEGFR; HER-2 (erbB-2); HER-3 (erbB-3); HER-4 (erbB-4), and IGF-IR.

Preferably, the methods further comprise administration of the cell surface receptor isoforms of this invention in combination with a therapeutically effective amount of an agent selected from the group consisting of: insulin, insulin-sensitizing agents, insulin secretogogues, and combinations thereof. Preferably, the insulin-sensitizing agent is selected from the group consisting of biguanides, metformin, thiazolidinediones (glitazones), and combinations thereof. Preferably, the insulin secretogogue is selected from the group consisting of sulfonylureas, meglitinides, and combinations thereof.

Pharmaceutical compositions. Additional preferred embodiments provide a pharmaceutical composition for treating a condition having an aspect related to, or associated with or characterized by altered IR expression or altered IR-mediated signaling at a cellular level, comprising, along with a pharmaceutically acceptable carrier or excipient, a cell surface receptor isoform such as Herstatin, or a variant thereof (e.g., a Int8 RBD polypeptide, or a variant thereof), that binds to the extracellular domain of a cellular target IR. Preferably, the condition is selected from the group consisting of insulin resistance syndrome, pre-diabetic conditions, metabolic syndrome, type 1 and type 2 diabetes, cardiac disease, diabetes-associated vascular disease, atherosclerosis, hypertension, diabetes-associated lipid metabolism disorders (dyslipidemia), obesity, critical illness, neurodegenerative disorders, and combinations thereof. In alternative related preferred embodiments, the targeted cell further expresses a target receptor selected from the group consisting of: EGFR (HER-1, erbB-1); ΔEGFR; HER-2 (erbB-2); HER-3 (erbB-3); HER-4 (erbB-4); IGF-IR, and combinations thereof. Preferably, the pharmaceutical composition further comprises an agent selected from the group consisting of: insulin, insulin-sensitizing agents, insulin secretogogues, and combinations thereof Preferably, the insulin-sensitizing agent is selected from the group consisting of biguanides, metformin, thiazolidinediones (glitazones), and combinations thereof. Preferably, the insulin secretogogue is selected from the group consisting of sulfonylureas, meglitinides, and combinations thereof.

Cell targeting. Yet further preferred embodiments provide methods and compositions for targeting a therapeutic agent to a cell expressing IR, comprising attaching the therapeutic agent to the cell surface receptor isoform, such as Herstatin, or to a variant thereof (e.g., a Int8 RBD polypeptide, or a variant thereof), that binds to the extracellular domain of a cellular target IR.

In related embodiments, the targeted cell further expresses a target receptor selected from the group consisting of: EGFR (HER-1, erbB-1); ΔEGFR; HER-2 (erbB-2); HER-3 (erbB-3); HER-4 (erbB-4); IGF-IR, and combinations thereof.

Preferably, in all of the above-described preferred embodiments, the Herstatin, or variant thereof, comprises a polypeptide selected from the group consisting of SEQ ID NO:2, or a fragment of SEQ ID NO:2 of about 80 to 419 contiguous residues in length, wherein the C-terminal 79 contiguous amino acids are present, wherein at least one N-linked glycosylation site is present, and wherein the polypeptide binds to the extracellular domain of insulin receptor with an affinity binding constant of at least 10⁸ M⁻¹. In particular aspects, the Herstatin, or variant thereof, comprises a sequence selected from the group consisting of SEQ ID NOS:32-42. Preferably, the Herstatin or variant thereof comprises SEQ ID NO:32. Preferably, the Herstatin or variant thereof consists of SEQ ID NO:32.

Preferably, the Int8 RBD polypeptide, or a variant thereof comprises a polypeptide selected from the group consisting of SEQ ID NO:1, or a fragment of SEQ ID NO:1 of about 50 to 79 contiguous residues in length, wherein the polypeptide binds to the extracellular domain of insulin receptor with an affinity binding constant of at least 10⁸ M⁻¹. In particular aspects, the Int8 RBD polypeptide, or a variant thereof, comprises a sequence selected from the group consisting of SEQ ID NOS:21-31. Preferably, the Int8 RBD polypeptide or variant thereof comprises SEQ ID NO:21. Preferably, the Int8 RBD polypeptide or variant thereof consists of SEQ ID NO:21.

### BRIEF DESCRIPTION OF THE DRAWINGS

FIG. 1 shows, according to particular aspects of the present invention as described in more detail in EXAMPLE II below, that Herstatin bound at nM concentrations to 3T3 cells over-expressing insulin receptor (IR), but not to 3T3 parental cells.

FIGS. 2A and 2B show, according to particular aspects as described in more detail in EXAMPLE III below, that Herstatin expression up-regulated IR expression and activation in MCF-7 cells.

FIGS. 3A and 3B show, according to particular aspects as described in more detail in EXAMPLE IV below, that in MCF-7 cells Herstatin expression substantially amplified insulin-stimulated ERK activation.

FIGS. 4A, 4B, 4C and 4D show, according to particular aspects as described in more detail in EXAMPLE V below, that Herstatin altered the expression of an array of proteins that are directly involved in insulin action.

FIG. 5 shows, according to particular aspects, that the EGFR inhibitor AS1478 does not affect insulin signaling.

FIG. 6 shows, according to particular aspects, that inhibition of the EGF receptor with an EGF receptor-specific inhibitor does not lead to an increase in insulin receptor.

### DETAILED DESCRIPTION OF THE INVENTION

Herstatin is an example of a cell surface receptor isoform, that may also be referred to as an alternative receptor product or an intron fusion protein, which functions as a receptor ligand, and functions as a secreted ligand that inhibits members of the EGF receptor family. Herstatin binds with high affinity to all members of the EGF receptor family, including EGFR/HER1/erbB1, HER2/neu/erbB2, HER3/erbB3, HER4/erbB4, and to ΔEGFR, and further binds to the IGF-IR.

The present invention discloses for the first time that the insulin receptor (IR) is a target of the cell surface receptor isoform, Herstatin, which specifically binds to the IR with nM affinity. According to preferred aspects of the present invention, Herstatin binds at nM concentrations to cell-surface IR, and further modulates insulin signaling in cells (e.g., MCF-7 human breast cancer cells, etc) expressing IR.

Herstatin is disclosed herein to alter expression of the IR and in particular to up-regulate basal IR expression by several-fold, and induce the accumulation of pro-IR.

Herstatin is further disclosed herein to modulate insulin activation. Herstatin stimulates insulin activation of the ERK pathway in a range of about 5- to about 80-fold, while having a more modest to little effect on insulin-stimulated (IR-mediated) activation of the P13K/Akt pathway.

Significantly, these changes in insulin signaling were shown herein to be accompanied by a decrease in IGF-IR expression in the range of about a 2- to about a 10-fold decrease, a decrease in the apparent serine phosphorylation state of IRS-1, and a slight decrease in IRS-2 levels as well as a decrease in apparent serine phosphorylation of IRS-2.

Therefore, preferred aspects of the present invention provide for uses of Herstatin in novel methods and compositions for treating a condition having an aspect related to, or associated with or characterized by altered IR expression or IR-mediated signal transduction.

The instant description and Examples, in various aspects, disclose the ability of Herstatin to modulate insulin action in cell models (e.g., a breast cancer cell model that consists of the well-characterized MCF-7 human breast cancer cell line, and two derivative clones that express human Herstatin from a stably transfected expression vector).

In particular aspects, Herstatin binding to cell-surface IR was investigated using IR-expressing 3T3 cells (IRA-3T3). Moreover, the effects of Herstatin on the expression and activation of the IR itself, and upon the expression and activation of the major signaling pathways that emanate from the activated insulin receptor (e.g., the ERK pathway and the P13K/Akt pathway) were investigated in MCF-7 and in Herstatin-expressing MCF-7 cells. All of the individual assays were repeated a minimum of three times with similar, if not identical, results, and many of the findings have been replicated and confirmed in experiments with an independent Herstatin-expressing MCF-7 clone.

According to preferred aspects of the present invention, Herstatin upregulates IR expression and IR-mediated signal transduction (e.g., substantially (>40-fold) stimulating insulin activation of the ERK pathway). Therefore, Herstatin and/or RBD Int8 polypeptides, and Herstatin- and/or RBD Int8 polypeptide-based agents (e.g., conjugates with drugs, toxins, radionuclides, etc.) have utility as therapeutic agents for treatment of diseases or conditions having an aspect related to, or associated with or characterized by altered IR expression or altered IR-mediated signaling at a cellular level (e.g., insulin resistance syndrome, pre-diabetic conditions, metabolic syndrome, type 1 and type 2 diabetes, cardiac disease, diabetes-associated vascular disease, atherosclerosis, hypertension, diabetes-associated lipid metabolism disorders (dyslipidemia), obesity, critical illness, neurodegenerative disorders, and combinations thereof).

Preferred aspects provide novel methods and compositions for treating cellular insulin resistance (for discussion of insulin resistance see, e.g., Alsheikh-Ali & Karas, Amer J Cardiology, 93:1417-8, 2004; Ovalle & Fernando, Southern Med J., 95:1188-94, 2002; and Zangeneh et al., Mayo Clinic Proc. 78:471-479, 2003).

According to additional preferred aspects, Herstatin and/or Herstatin-based agents can be used to target IR-expressing cells and/or modulate IR-mediated signaling.

### DEFINITIONS

“Herstatin,” an example of a cell surface receptor isoform (also referred to as an intron fusion protein) refers to the polypeptides of SEQ ID NO:2 (including SEQ ID NOS:32-42), and additionally includes functional (e.g., target receptor-binding) variants (including conservative amino acid sequence variants as described herein), fragments, muteins, derivatives and fusion proteins thereof.

As used herein, an isoform of a cell surface receptor (also referred to herein as a CSR isoform), such as an isoform of a receptor tyrosine kinase, refers to a receptor that lacks a domain or portion thereof sufficient to alter or modulate a biological activity of the receptor or modulate a biological activity compared to a wildtype and/or predominant form of the receptor. A CSR isoform refers to a receptor that lacks a domain or portion of a domain sufficient to alter or modulate a biological activity of the receptor, for example the insulin receptor. Generally, a biological activity is altered in an isoform at least 0.1, 0.5, 1, 2, 3, 4, 5, or 10-fold compared to a wildtype and/or predominant form of the receptor. Typically, a biological activity is altered 10-, 20-, 50-, 100- or 1000-fold or more. With reference to an isoform, alteration of activity refers to difference in activity between the particular isoform, which is shortened, compared to the unshortened form of the receptor. Alteration of biological activity includes an enhancement or a reduction of activity. In particular embodiments, alteration of a biological activity is a reduction in the activity. In particular embodiments, an alteration of a biological activity is a reduction in biological activity, and the reduction can be at least 0.1 0.5 1, 2, 3, 4, 5, or 10-fold compared to a wildtype and/or predominant form of the receptor. Typically, a biological activity is reduced 5, 10, 20, 50, 100 or 1000-fold or more. Reference herein to a CSR isoform with altered activity refers to the alteration in an activity by virtue of the different structure or sequence of the CSR isoform compared to a cognate receptor.

Reference herein to modulating the activity of a target cell surface receptor means that a CSR isoform interacts in some manner with the target receptor and activity, such as ligand binding or dimerization or other signal-transduction-related activity is altered.

Intron fusion proteins (IFPs) are exemplary CSR isoforms. IFPs, for purposes herein include natural and combinatorial IFPs. A natural IFP refers to a polypeptide that is encoded by an alternatively spliced RNA that contains one or more amino acids encoded by an intron operatively linked to one or more portions of the polypeptide encoded by one or more exons of a gene. Alternatively spliced mRNA is one that is isolated or is one that can be prepared synthetically by joining splice donor and acceptor sites in a gene. A natural IFP contains one or more amino acids and/or one or more stop codons encoded by an intron sequence. A combinatorial IFP refers to a polypeptide that is shortened compared to a wildtype or predominant form of a polypeptide. Typically, the shortening removes one or more domains or a portion thereof from a polypeptide such that a biological activity is altered. Combinatorial IFPs often mimic a natural IFP in that one or more domains or a portion thereof that is/are deleted in a natural IFP derived from the same gene sequence or derived from a gene sequence in a related gene family.

As used herein, natural with reference to IFP, refers to any protein, polypeptide or peptide or fragment thereof (by virtue of the presence of the appropriate splice acceptor/donor sites) that is encoded within the genome of an animal and/or is produced or generated in an animal or that could be produced from a gene. Natural IFPs include allelic variant. IFPs can be modified post-translationally.

“RBD Int8 polypeptide” refers to the polypeptides of SEQ ID NO:1 (including SEQ ID NOS:21-31), and additionally includes functional (e.g., target receptor-binding) variants (including conservative amino acid sequence variants as described herein), fragments, muteins, derivatives and fusion proteins thereof.

“Mutant RBD Int8 polypeptide” or “mutant Int8 RBD polypeptide” refers to the intron 8-encoded receptor binding domain variants (with an Arg to Ile mutation at residue 31 thereof) of SEQ ID NO:3), and additionally includes functional (e.g., target receptor non-binding) variants (including conservative amino acid sequence variants as described herein), fragments, muteins, derivatives and fusion proteins thereof. Representative, corresponding Herstatin variants (Arg to Ile mutation at residue 371) are given as SEQ ID NO:4.

“EGFR,” “HER-1” or “erbB-1” refer to the art-recognized human epidermal growth factor receptor, erbB-1 (cDNA: NM_{—}005228, SEQ ID NO:5; protein: NP_{—}005219, SEQ ID NO:6), and including Herstatin-, and/or Int8 RBD polypeptide-binding variants thereof.

“ΔEGFR” refers to the art-recognized receptor, ΔEGFR (cDNA: SEQ ID NO:7; protein: SEQ ID NO:8) (see Ekstrand et al., PNAS 89:4309-4313, 1992; and Nishikawa et al., PNAS 91:7727-7731, 1994) (comprising a deletion in the ECD; cDNA positions 275 through 1075, corresponding to exons 2-7 of the EGFR gene), and including Herstatin-, and/or Int8 RBD polypeptide-binding variants thereof.

“HER-2” or “erbB-2” refers to the art-recognized human receptor, erbB-2 (cDNA: NM_{—}004448, SEQ ID NO:9; protein: NP_{—}004439, SEQ ID NO:10), and including Herstatin-, and/or Int8 RBD polypeptide-binding variants thereof.

“HER-3” or “erbB-3” refers to the art-recognized human receptor, erbB-3 (cDNA: NM_{—}001982, SEQ ID NO:11; protein: NP_{—}001973, SEQ ID NO:12), and including Herstatin-, and/or Int8 RBD polypeptide-binding variants thereof.

The phrase “mutant form of HER-3” refers to a HER-3 protein having a substitution of Glu for Gly in the ectodomain of HER-3 corresponding to a single point mutation at nucleotide position 1877 (“a” instead of “g” at this position), resulting in substitution of Glu instead of Gly at residue position 560) (cDNA: SEQ ID NO:13; protein: SEQ ID NO:14).

“HER-4” or “erbB-4” refers to the art-recognized human receptor, erbB-4 (cDNA: NM_{—}005235, SEQ ID NO:15; protein: NP_{—}005226, SEQ ID NO:16), and including Herstatin-, and/or Int8 RBD polypeptide-binding variants thereof.

“IGF-IR” refers to the art recognized insulin-like growth factor I receptor (cDNA: NM_{—}000875, SEQ ID NO:17; protein: NP_{—}000866, SEQ ID NO:18), and including Herstatin-, and/or Int8 RBD polypeptide-binding variants thereof.

“Insulin receptor” or IR refers to the art-recognized insulin receptor (cDNA: NM_{—}000208, SEQ ID NO:19; protein: NP_{—}000199, SEQ ID NO:20), and including Herstatin-, and/or Int8 RBD polypeptide-binding variants thereof.

TABLE 1
Summary of key SEQ ID NOS and accession numbers:

| MOLECULE | cDNA | PROTEIN |
|---|---|---|
| RBD Int8 polypeptide(s)) | | SEQ ID NO: 1 |
| Herstatin(s) | | SEQ ID NO: 2 |
| | | SEQ ID NOS: 32-42 |
| Mutant Int8 RBD | | SEQ ID NO: 3 |
| polypeptide(s) | | SEQ ID NOS: 21-31 |
| Mutant Herstatin(s) | | SEQ ID NO: 4 |
| EGFR (HER-1 or erbB-1) | SEQ ID NO: 5 (NM_005228) | SEQ ID NO: 6 (NP_005219) |
| ΔEGFR | SEQ ID NO: 7 | SEQ ID NO: 8 |
| HER-2 (erbB-2) | SEQ ID NO: 9 (NM_004448) | SEQ ID NO: 10 (NP_004439) |
| HER-3 (erbB-3) | SEQ ID NO: 11 (NM_001982) | SEQ ID NO: 12 (NP_001973) |
| Mutant form of HER-3 | SEQ ID NO: 13 | SEQ ID NO: 14 |
| HER-4 (erbB-4) | SEQ ID NO: 15 (NM_005235) | SEQ ID NO: 16 (NP_005226) |
| IGF-IR | SEQ ID NO: 17 (NM_000875) | SEQ ID NO: 18 (NP_000866) |
| Insulin receptor (IR) | SEQ ID NO: 19 (NM_000208) | SEQ ID NO: 20 |
| | | (NP_000199.1) |

### Cell Surface Receptor (CSR) Isoforms

Provided herein are cell surface receptor (CSR) isoforms (including intron fusion proteins; IFPs) having the novel biological activity of altering IR expression or altered IR mediated signaling. The CSR isoforms differ from the cognate receptors in that there are insertions and/or deletions, and the resulting CSR isoforms exhibit a difference in one or more activities or functions compared to the cognate receptor. Such differences include, for example elimination of all or part of a transmembrane domain, and/or a change in a biological activity of the CSR (e.g., as disclosed herein, the ability to modulate insulin receptor (IR) expression or IR-mediated signaling). The CSR isoforms provided herein can be used for modulating the activity of a cell surface receptor (e.g., the IR). They also can be used as targeting agents (e.g., targeting IR) for delivery of molecules, such as drugs or toxins or nucleic acids, to targeted cells or tissues.

A CSR isoform refers to a receptor that lacks a domain or portion of a domain sufficient to alter a biological activity (e.g., an activity with respect to the IR). Thus, an isoform may differ from a wildtype and/or predominant form of the receptor, in that it lacks one or more biological activities of the receptor. Additionally, CSR isoforms can contain a new domain and/or biological function as compared to a wildtype and/or predominant form of the receptor. For example, intron-encoded amino acids can introduce a new domain or portion thereof into a CSR isoform. Biological activities that can be altered (or gained) include, but are not limited to, protein-protein interactions such as dimerization, multimerization and complex formation, specificity and/or affinity for ligand, cellular localization and relocalization, membrane anchoring, enzymatic activity such as kinase activity, response to regulatory molecules including regulatory proteins, cofactors, and other signaling molecules, such as in a signal transduction pathway. Generally, a biological activity is altered in an isoform at least 0.1, 0.5, 1, 2, 3, 4, 5, or 10-fold as compared to a wildtype and/or predominant form of the receptor. Typically, a biological activity is altered 10, 20, 50, 100 or 1000-fold or more. For example, an isoform can be reduced with respect to a particular biological activity.

CSR isoforms can also modulate an activity of a wildtype and/or predominant form of the cognate receptor. For example, a CSR isoform can interact directly or indirectly with a CSR isoform and modulate a biological activity of the cognate receptor. Biological activities that can be altered include, but are not limited to, protein-protein interactions such as dimerization, multimerization and complex formation, specificity and/or affinity for ligand, cellular localization and relocalization, membrane anchoring, enzymatic activity such as kinase activity, response to regulatory molecules including regulatory proteins, cofactors, and other signaling molecules, such as in a signal transduction pathway.

A CSR isoform can interact directly or indirectly with a cell surface receptor to cause or participate in a biological effect, such as by modulating a biological activity of the cell surface receptor (e.g., in the instant case, the IR). A CSR isoform also can interact independently of a cell surface receptor to cause a biological effect, such as by initiating or inhibiting a signal transduction pathway. For example, a CSR isoform can initiate a signal transduction pathway and enhance or promote cellular metabolism. In another example, a CSR isoform can interact with the cell surface receptor as a ligand, causing a biological effect for example by inhibiting a signal transduction pathway that can promote or alter a cellular response to insulin. Hence, the isoforms provided herein can function as cell surface receptor ligands in that they interact with the targeted receptor in the same manner that a cognate ligand interacts with and alters receptor activity. The isoforms can bind as a ligand, but not necessarily to the ligand binding site, and can serve to block receptor dimerization. They act as ligands in the sense that they interact with the receptor. The CSR isoforms also can act by binding to ligands for the receptor and/or by preventing receptor activities, such as dimerization.

For example, a CSR isoform can compete with a CSR for ligand binding. A CSR isoform can act as a dominant negative inhibitor, for example, when complexed with a CSR. A CSR isoform can act as a dominant negative inhibitor or as a competitive inhibitor of a CSR, for example, by complexing with a CSR isoform and altering the ability of the CSR to multimerize (e.g, dimerize or trimerize) with other CSRs. A CSR isoform can compete with a CSR for interactions with other polypeptides and cofactors in a signal transduction pathway.

The cell surface isoforms and families of isoforms provided herein include, for example, isoforms of the HER-2 receptor (e.g., Herstatin), IR, etc. Pharmaceutical compositions containing one or more different CSR isoforms are provided. Also provided are methods of treatment of diseases and conditions by administering the pharmaceutical compositions or delivering a CSR isoform, such by administering the isoform protein (polypeptide, etc), and/or by administration of a vector that encodes the isoform. Administration, by either means, can be effected in vivo or ex vivo. Also provided are methods for expressing, isolating and formulating CSR isoforms.

Herstatin and/or RBD Int8 Polyepeptides and Therapeutic Agents

In preferred aspects, the present invention provides for Herstatin (e.g., the sequences of SEQ ID NO:2) and polypeptides thereof that bind to a insulin receptor (IR) as a target receptor (specifically, or in addition to the known targets: EGFR, HER-2, HER-3, DEGFR, HER-4 and IGF-IR). Also provided are RBD Int8 polypeptides (e.g., the sequences of SEQ ID NO:1) and receptor-binding polypeptides thereof that bind to a insulin receptor as a target receptor (specifically, or in addition to the known targets EGFR, HER-2, HER-3, DEGFR, HER-4 and IGF-IR).

Preferably, the Herstatin and/or RBD Int8 polypeptides comprise an amino acid sequence of SEQ ID NO:1 (or of SEQ ID NO:1 having from 1, to about 3, to about 5, to about 10, or to about 20 conservative amino acid substitutions), or a fragment of a sequence of SEQ ID NO:1 (or a fragment of SEQ ID NO:1 having from 1, to about 3, to about 5, to about 10, or to about 20 conservative amino acid substitutions) of about 50 to 79 contiguous residues in length, wherein the polypeptide binds to the extracellular domain (ECD) of a target receptor (e.g., EGFR, HER-2, HER-3, DEGFR, HER-4, IGF-IR and IR (as disclosed herein)) with an affinity binding constant of at least 10⁷ M⁻¹, at least 5×10⁷ M⁻¹, or at least 10⁸ M⁻¹. Preferably, the Herstatin and/or RBD Int8 polypeptide is from about 69 to 79 contiguous residues in length, with a IR affinity binding constant of at least 10⁷ M⁻¹, at least 5×10⁷ M⁻¹, or at least 10⁸ M⁻¹ (similar to the respective binding constants associated with the known EGFR, HER-2, HER-3, DEGFR, HER-4 and IGF-IR target receptors). Preferably, Herstatin and/or RBD Int8 polypeptide comprises a sequence of SEQ ID NO:1, or a conservative amino acid substitution variant thereof. In particular aspects, the Int8 RBD polypeptide, or a variant thereof, comprises a sequence selected from the group consisting of SEQ ID NOS:21-31. Preferably, the Int8 RBD polypeptide or variant thereof comprises SEQ ID NO:21. Preferably, the Int8 RBD polypeptide or variant thereof consists of SEQ ID NO:21.

Preferably, the Herstatin and/or RBD Int8 polypeptides comprise an amino acid sequence of SEQ ID NO:2 (or of SEQ ID NO:2 having from 1, to about 3, to about 5, to about 10, or to about 20 conservative amino acid substitutions), or a fragment of a sequence of SEQ ID NO:2 (or a fragment of SEQ ID NO:2 having from 1, to about 3, to about 5, to about 10, or to about 20 conservative amino acid substitutions) of about 80 to 419 contiguous residues in length, wherein the C-terminal 79 contiguous amino acids are present, and wherein the polypeptide binds to the extracellular domain (ECD) of a IR with an affinity binding constant of at least 10⁷ M⁻¹, at least 5×10⁷ M⁻¹, or at least 10⁸ M⁻¹ (similar to the respective binding constants associated with the known EGFR, HER-2, HER-3, DEGFR, HER-4 and IGF-IR target receptors). Preferably, the Herstatin and/or RBD Int8 polypeptide is from about 350 to 419 contiguous residues in length, wherein the polypeptide binds to the extracellular domain (ECD) of a IR with an affinity binding constant of at least 10⁷ M⁻¹, at least 5×10⁷ M⁻¹, or at least 10⁸ M⁻¹ (similar to the respective binding constants associated with the known EGFR, HER-2, HER-3, DEGFR, HER-4 and IGF-IR target receptors). Preferably, comprises a sequence of SEQ ID NO:2, or a conservative amino acid substitution variant thereof. In particular aspects, the Herstatin, or variant thereof, comprises a sequence selected from the group consisting of SEQ ID NOS:32-42. Preferably, the Herstatin or variant thereof comprises SEQ ID NO:32. Preferably, the Herstatin or variant thereof consists of SEQ ID NO:32.

### Biologically Active Variants

Variants of Herstatin and/or RBD Int8 polypeptide have substantial utility in various aspects of the present invention. Variants can be naturally or non-naturally occurring. Naturally occurring variants are found in humans or other species and comprise amino acid sequences which are substantially identical to the amino acid sequences shown in SEQ ID NO:1 or SEQ ID NO:2, and include natural sequence polymorphisms. Species homologs of the protein can be obtained using subgenomic polynucleotides of the invention, as described below, to make suitable probes or primers for screening cDNA expression libraries from other species, such as mice, monkeys, yeast, or bacteria, identifying cDNAs which encode homologs of the protein, and expressing the cDNAs as is known in the art.

Non-naturally occurring variants which retain substantially the same biological activities as naturally occurring protein variants, including the target RBD activity and the modulation of target receptor signaling activity, are also included here. Preferably, naturally or non-naturally occurring variants have amino acid sequences which are at least 85%, 90%, or 95% identical to the amino acid sequence shown in SEQ ID NOS:1 or 2. More preferably, the molecules are at least 98% or 99% identical. Percent identity is determined using any method known in the art. A non-limiting example is the Smith-Waterman homology search algorithm using an affine gap search with a gap open penalty of 12 and a gap extension penalty of 1. The Smith-Waterman homology search algorithm is taught in Smith and Waterman, Adv. Appl. Math. 2:482-489, 1981.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R..§§.1.821-1.822, abbreviations for amino acid residues are shown in Table 1:

TABLE 1
Table of Correspondence

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Praline |
| K | Lys | Lysine |
| H | His | Histidine |
| Q | Gln | Glutamine |
| E | Glu | glutamic acid |
| Z | Glx | Glu and/or Gln |
| W | Trp | Tryptophan |
| R | Arg | Arginine |
| D | Asp | aspartic acid |
| N | Asn | Asparagines |
| B | Asx | Asn and/or Asp |
| C | Cys | Cysteine |
| X | Xaa | Unknown or other |

It should be noted that all amino acid residue sequences represented herein by a formula have a left to right orientation in the conventional direction of amino-terminus to carboxyl-terminus. In addition, the phrase “amino acid residue” is defined to include the amino acids listed in the Table of Correspondence and modified and unusual amino acids, such as those referred to in 37 C.F.R..§§ 1.821-1.822, and incorporated herein by reference. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

Guidance in determining which amino acid residues can be substituted, inserted, or deleted without abolishing biological or immunological activity can be found using computer programs well known in the art, such as DNASTAR™ software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224).

Such substitutions may be made in accordance with those set forth in TABLE 2 as follows:

TABLE 2

| Original | Conservative |
|---|---|
| residue | substitution |
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with other known conservative (or non-conservative) substitutions.

Variants of the Herstatin and/or RBD Int8 polypeptide disclosed herein include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

A subset of mutants, called muteins, is a group of polypeptides in which neutral amino acids, such as serines, are substituted for cysteine residues which do not participate in disulfide bonds. These mutants may be stable over a broader temperature range than native secreted proteins (Mark et al., U.S. Pat. No. 4,959,314).

Preferably, amino acid changes in the Herstatin and/or RBD Int8 polypeptide variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting secreted protein or polypeptide variant. Properties and functions of Herstatin and/or RBD Int8 polypeptide protein or polypeptide variants are of the same type as a protein comprising the amino acid sequence encoded by the nucleotide sequences shown in SEQ ID NO:1 or 2, although the properties and functions of variants can differ in degree.

Herstatin and/or RBD Int8 polypeptide variants include glycosylated forms, aggregative conjugates with other molecules, and covalent conjugates with unrelated chemical moieties (e.g., pegylated molecules). Herstatin and/or RBD Int8 polypeptide variants also include allelic variants (e.g., polymorphisms), species variants, and muteins. Truncations or deletions of regions which do not preclude functional activity of the proteins are also variants. Covalent variants can be prepared by linking functionalities to groups which are found in the amino acid chain or at the N- or C-terminal residue, as is known in the art.

It will be recognized in the art that some amino acid sequence of the Herstatin and/or RBD Int8 polypeptides of the invention can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of binding to cell surface receptors (Ostade et al., Nature 361:266-268, 1993). Thus, the Herstatin and/or RBD Int8 polypeptides of the present invention may include one or more amino acid substitutions, deletions or additions, either from natural mutations or human manipulation.

Of particular interest are substitutions of charged amino acids with another charged amino acid and with neutral or negatively charged amino acids. The latter results in proteins with reduced positive charge to improve the characteristics of the disclosed protein. The prevention of aggregation is highly desirable. Aggregation of proteins not only results in a loss of activity but can also be problematic when preparing pharmaceutical formulations, because they can be immunogenic (Pinckard et al., Clin. Exp. Immunol. 2:331-340, 1967; Robbins et al., Diabetes 36:838-845, 1987; Cleland et al., Crit. Rev. Therapeutic Drug Carrier Systems 10:307-377, 1993).

Amino acids in the Herstatin and/or RBD Int8 polypeptides of the present invention that are essential for function can be identified by methods known in the art, such as site-directed mutagenesis or alanine-scanning mutagenesis (Cunningham and Wells, Science 244:1081-1085, 1989). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith et al., J. Mol. Biol. 224:899-904, 1992 and de Vos et al. Science 255:306-312,1992).

As indicated, changes are preferably of a minor nature, such as conservative amino acid substitutions that do not significantly affect the folding or activity of the protein. Of course, the number of amino acid substitutions a skilled artisan would make depends on many factors, including those described above. Generally speaking, the number of substitutions for any given Herstatin and/or RBD Int8 polypeptide will not be more than 50, 40, 30, 25, 20, 15, 10, 5 or 3.

In addition, pegylation of Herstatin and/or RBD Int8 polypeptides and/or muteins is expected to provide such improved properties as increased half-life, solubility, and protease resistance. Pegylation is well known in the art.

### Fusion Proteins

Fusion proteins comprising proteins or polypeptide fragments of Herstatin and/or RBD Int8 polypeptide can also be constructed. Fusion proteins are useful for generating antibodies against amino acid sequences and for use in various targeting and assay systems. For example, fusion proteins can be used to identify proteins which interact with a Herstatin and/or RBD Int8 polypeptide of the invention or which interfere with its biological function. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence can be used.

A fusion protein comprises two protein segments fused together by means of a peptide bond. Amino acid sequences for use in fusion proteins of the invention can be utilize the amino acid sequence shown in SEQ ID NOS:1 or 2 or can be prepared from biologically active variants of SEQ ID NOS:1 or 2, such as those described above. The first protein segment can include of a full-length Herstatin and/or RBD Int8 polypeptide.

Other first protein segments can consist of about 50 to about 79 contiguous amino acids from SEQ ID NO:1, or, with respect to SEQ ID NO:2, from about 80 to 419 contiguous residues in length, wherein the C-terminal 79 contiguous amino acids of SEQ ID NO:2 are present, or from about 350 to 419 contiguous residues in length wherein the C-terminal 79 contiguous amino acids of SEQ ID NO:2 are present.

The second protein segment can be a full-length protein or a polypeptide fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions.

These fusions can be made, for example, by covalently linking two protein segments or by standard procedures in the art of molecular biology. Recombinant DNA methods can be used to prepare fusion proteins, for example, by making a DNA construct which comprises a coding region for the protein sequence of SEQ ID NOS:1 or 2 in proper reading frame with a nucleotide encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

### Cell Targeting

According to additional preferred aspects of the present invention, cell surface receptor isoforms such as Herstatin- and/or RBD Int8 polypeptide-based agents can be used to target insulin receptor (IR) on cells (e.g., insulin-resistant cells, IR-expressing cells involved with some aspect of glucose regulation or metabolism, cancer cells, etc.). Herstatin- and/or RBD Int8 polypeptide-based agents can be used to deliver a locally acting biological agent that will affect the targeted cell.

IR, in the context of the inventive targeting, is expressed on the surface of cells and is accessible (specifically, or in addition to at least one of the other known Herstatin targets: EGFR; HER-2; HER-3; HER-4, ΔEGFR and IGF-IR) to exogenous molecules. For example, where IR is present at higher levels on particular IR-bearing cells (e.g., adipocytes, hepatocytes, skeletal muscle cells, pancreatic beta cells, brain/nerve cells, etc) as compared to other cells, they can be utilized as preferential targets for systemic Herstatin- and/or RBD Int8 polypeptide-based agents and therapies. The differential expression of the target receptor (e.g., IR) enables the specificity of Herstatin- and/or RBD Int8 polypeptide-based agents-based therapy. Herstatin- and/or RBD Int8 polypeptide-based agents (e.g., drugs, cytoxic agents, labeling agents, etc.) directed against the target receptor preferentially affect the targeted cell over normal tissue. For example, a Herstatin- or RBD Int8 polypeptide-drug conjugate that binds a IR present predominantly on particular cells (e.g., adipocytes, hepatocytes, skeletal muscle cells, pancreatic beta cells, brain/nerve cells, etc) would be expected to selectively affect those cells within a treated individual. Preferably, the target receptor is accessible to the Herstatin- and/or RBD Int8 polypeptide-based agent, and is found in substantially greater concentrations on the targeted cells (e.g., adipocytes, hepatocytes, skeletal muscle cells, pancreatic beta cells, brain/nerve cells, etc) relative to other cells that don't express IR or that express IR at relatively low levels.

Therefore, the present invention includes Herstatin- and/or RBD Int8 polypeptide-based agents specific to one or more of the target receptors (e.g., IR) that will enable or facilitate therapeutic treatments relating to, for example, adipocytes, hepatocytes, skeletal muscle cells, pancreatic beta cells, brain cells, etc.

In particular aspects, Herstatin- and/or RBD Int8 polypeptides are conjugated or coupled to drugs, or to toxins.

In alternate embodiments, Herstatin- and/or RBD Int8 polypeptides are conjugated or coupled to radionuclides.

Additional embodiments provide for Herstatin- and/or RBD Int8 polypeptide-coated liposomes that contain one or more biologically active compounds.

In preferred embodiments, Herstatin-mediated targeting is used to deliver drugs or other agents to adipocytes, hepatocytes, skeletal muscle cells, pancreatic beta cells, brain cells, and combinations thereof.

In alternate aspects, targeted binding of an Herstatin- and/or RBD Int8 polypeptide-agent to a cell is sufficient to modulate IR-mediated signaling, inhibit or alter growth (e.g., cytostatic effects) or even kill the target cell (cytotoxic effects) if so desired. The mechanism of these activities may vary, but may involve Herstatin- and/or RBD int8 polypeptide-dependent receptor activation, changes in receptor expression, cell-mediated cytotoxicity, activation of apoptosis, inhibition of ligand-receptor function, or provide a signal for complement fixation. In fact, Herstatin- and/or RBD Int8 polypeptide-agents may exhibit one or several such activities. In particular aspects, Herstatin- and/or RBD Int8 polypeptide-agents are cytostatic, but not cytotoxic. In particular embodiments, Herstatin- and/or RBD Int8 polypeptide-agents bind to target receptors (e.g., IR, EGFR (HER-1, erbB-1); HER-2 (erbB-2); HER-3 (erbB-3); HER-4 (erbB-4), ΔEGFR or IGF-IR), and modulate signaling and cellular metabolism, or are either cytoxic or cytostatic, etc.

In additional embodiments, Herstatin- and/or RBD Int8 polypeptide-agents are conjugated or coupled to a diverse array of compounds which include, but are not limited to proteins, drugs, toxins or cytotoxic agents, cytostatic agents, radionuclides, apoptotic factors (Wuest et al. 2002), anti-angiogenic compounds or other biologically active compounds which will affect cellular signaling or metabolism, inhibit the growth of or even kill the target cell or tissue. For example, cytotoxic or cytostatic agents include, but are not limited to, diphtheria toxin and Pseudomonas exotoxin (Kreitman 2001 a; Kreitman 2001 b), ricin (Kreitman 2001 a), gelonin, doxorubicin (Ajani et al. 2000) and its derivatives, iodine-131, yttrium-90 (Witzig 2001), indium-111 (Witzig 2001), RNase (Newton and Ryback 2001), calicheamicin (Bernstein 2000), apoptotic agents, and antiangiogenic agents (Frankel et al. 2000; Brinkmann et al. 2001; Garnett 2001). According to particular aspects of the present invention, Herstatin- and/or RBD Int8 polypeptides coupled to these compounds are used to adversely affect cells displaying one or more target receptors (e.g., IR, EGFR (HER-1, erbB-1); HER-2 (erbB-2); HER-3 (erbB-3); HER-4 (erbB-4), ΔEGFR or IGF-IR).

Toxins can also be targeted to specific cells by incorporation of the toxin into Herstatin- and/or RBD Int8 polypeptide-coated liposomes. The Herstatin- and/or RBD Int8 polypeptide-based agent directs the liposome to the target cell where the bioactive compound is released. For example, cytotoxins in Herstatin- and/or RBD Int8 polypeptide-coated liposomes are used to treat cancer. In alternate embodiments, these targeted liposomes are loaded with DNA encoding bioactive polypeptides (e.g., inducible nitric oxide synthase; Khare et al. 2001).

Prodrugs or enzymes can also be delivered to targeted cells by specific Herstatin- and/or RBD Int8 polypeptide-agents. In this case the Herstatin conjugate consists of a Herstatin- and/or RBD Int8 polypeptide-based agent coupled to a drug that can be activated once the polypeptide agent binds the target cell. Examples of this strategy using antibodies have been reviewed (Denny 2001; Xu and McLeod 2001).

Therefore, in particular embodiments, Herstatin- and/or RBD Int8 polypeptide-prodrug/enzyme conjugates targeted to one or more target receptors (e.g., IR, EGFR (HER-1, erbB-1); HER-2 (erbB-2); HER-3 (erbB-3); HER-4 (erbB-4), ΔEGFR or IGF-IR) have utility for the treatment of, for example, cancer and other treatable conditions discussed herein.

The specificity and high affinity of the Herstatin- and/or RBD Int8 polypeptide-based agents makes them ideal candidates for delivery of toxic agents to a specific subset of cellular targets. Preferably, one or more target receptors (e.g., IR, EGFR (HER-1, erbB-1); HER-2 (erbB-2); HER-3 (erbB-3); HER-4 (erbB-4), ΔEGFR or IGF-IR) are present at higher levels on the target cells (e.g., cancer, tumor cells) than on non-cancer cells.

As used herein, a composition refers to any mixture. It can be a solution, a suspension, liquid, powder, a paste, aqueous, non-aqueous or any combination thereof.

As used herein, a combination refers to any association between or among two or more items. The combination can be two or more separate items, such as two compositions or two collections, can be a mixture thereof, such as a single mixture of the two or more items, or any variation thereof.

As used herein, a pharmaceutical effect refers to an effect observed upon administration of an agent intended for treatment of a disease or disorder or for amelioration of the symptoms thereof.

As used herein, treatment means any manner in which the symptoms of a condition, disorder or disease or other indication, are ameliorated or otherwise beneficially altered.

As used herein therapeutic effect means an effect resulting from treatment of a subject that alters, typically improves or ameliorates the symptoms of a disease or condition or that cures a disease or condition. A therapeutically effective amount refers to the amount of a composition, molecule or compound which results in a therapeutic effect following administration to a subject.

In particular aspects, a therapeutic effect may also encompass prophylaxis of symptoms of a condition.

As used herein, the term “subject” refers to animals, including mammals, such as human beings. As used herein, a patient refers to a human subject.

As used herein, the phrase “associated with” or “characterized by” refers to certain biological aspects such as expression of a receptor or signaling by a receptor that occurs in the context of a disease or condition. Such biological aspects may or may not be causative or integral to the disease or condition but merely an aspect of the disease or condition.

As used herein, a biological activity refers to a function of a polypeptide including but not limited to complexation, dimerization, multimerization, receptor-associated kinase activity, receptor-associated protease activity, phosphorylation, dephosphorylation, autophosphorylation, ability to form complexes with other molecules, ligand binding, catalytic or enzymatic activity, activation including auto-activation and activation of other polypeptides, inhibition or modulation of another molecule's function, stimulation or inhibition of signal transduction and/or cellular responses such as cell proliferation, migration, differentiation, and growth, degradation, membrane localization, membrane binding, and oncogenesis. A biological activity can be assessed by assays described herein and by any suitable assays known to those of skill in the art, including, but not limited to in vitro assays, including cell-based assays, in vivo assays, including assays in animal models for particular diseases.

### Pharmaceutical Compositions and Therapeutic Uses

Pharmaceutical compositions of the invention comprise a cell surface receptor isoform such as Herstatin and/or RBD Int8 polypeptides, or Herstatin- and/or RBD Int8 polypeptide-based agents of the claimed invention in a therapeutically effective amount. The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation is determined by routine experimentation and is within the judgment of the clinician. For purposes of the present invention, an effective dose will generally be from about 0.01 mg/kg to 50 mg/kg or 0.05 mg/kg to about 10 mg/kg of the Herstatin and/or RBD Int8 polypeptide constructs in the individual to which it is administered. A non-limiting example of a pharmaceutical composition is a composition that either enhances or diminishes signaling mediated by the inventive target receptors (e.g., IR, EGFR, HER-2, HER-3, ΔEGFR, HER-4 and IGF-IR). Where such signaling modulates a disease-related process, modulation of the signaling would be the goal of the therapy.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles. Typically, the therapeutic compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., New Jersey, 1991).

### Delivery Methods

Once formulated, the compositions of the invention can be administered (as proteins/polypeptides, or in the context of expression vectors for gene therapy) directly to the subject or delivered ex vivo, to cells derived from the subject (e.g., as in ex vivo gene therapy). Direct delivery of the compositions will generally be accomplished by parenteral injection, e.g., subcutaneously, intraperitoneally, intravenously or intramuscularly, myocardial, intratumoral, peritumoral, or to the interstitial space of a tissue. Other modes of administration include oral and pulmonary administration, suppositories, and transdermal applications, needles, and gene guns or hyposprays. Dosage treatment can be a single dose schedule or a multiple dose schedule.

Methods for the ex vivo delivery and reimplantation of transformed cells into a subject are known in the art and described in, for example, International Publication No. WO 93/14778. Examples of cells useful in ex vivo applications include, for example, stem cells, particularly hematopoetic, lymph cells, macrophages, dendritic cells, or tumor cells. Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, direct microinjection of the DNA into nuclei, and viral-mediated, such as adenovirus (and adeno-associated virus) or alphavirus, all well known in the art.

In a preferred embodiment, certain disorders (e.g., of proliferation, such as cancer, etc), can be amenable to treatment by administration of a therapeutic agent based on the provided polynucleotide or corresponding polypeptide. The therapeutic agent can be administered in conjunction with one or more other agents including, but not limited to, receptor-specific antibodies and/or other agents (e.g., insulin-sensitizing agents, chemotherapeutic agents, etc). Administered “in conjunction” includes administration at the same time, or within 1 day, 12 hours, 6 hours, one hour, or less than one hour, as the other therapeutic agent(s). The compositions may be mixed for co-administration, or may be administered separately by the same or different routes.

The dose and the means of administration of the inventive pharmaceutical compositions are determined based on the specific qualities of the therapeutic composition, the condition, age, and weight of the patient, the progression of the disease, and other relevant factors. For example, administration of polynucleotide therapeutic compositions agents of the invention includes local or systemic administration, including injection, oral administration, particle gun or catheterized administration, and topical administration. The therapeutic polynucleotide composition can contain an expression construct comprising a promoter operably linked to a polynucleotide encoding, for example, about 80 to 419 (or about 350 to 419) contiguous amino acids of SEQ ID NO:2. Various methods can be used to administer the therapeutic composition directly to a specific site in the body. For example, an abnormal tissue, or small metastatic lesion is located and the therapeutic composition injected several times in several different locations within the body of the tissue, or tumor. Alternatively, arteries which serve a tissue or tumor are identified, and the therapeutic composition injected into such an artery, in order to deliver the composition directly into the tumor. A tissue or tumor that has a necrotic center is aspirated and the composition injected directly into the now empty center of the tissue or tumor. X-ray imaging is used to assist in certain of the above delivery methods.

Herstatin and/or RBD Int8 polypeptide-mediated targeted delivery of therapeutic agents to specific tissues can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. (USA) (1990) 87:3655; Wu et al., J. Biol. Chem. (1991) 266:338.

For gene therapy, therapeutic compositions containing a polynucleotide are administered in a range of about 100 ng to about 200 mg of DNA for local administration in a gene therapy protocol. Concentration ranges of about 500 ng to about 50 mg, about 1 mg to about 2 mg, about 5 mg to about 500 mg, and about 20 mg to about 100 mg of DNA can also be used during a gene therapy protocol. Factors such as method of action (e.g., for enhancing or inhibiting levels of the encoded gene product) and efficacy of transformation and expression are considerations which will affect the dosage required for ultimate efficacy of the subgenomic polynucleotides. Where greater expression is desired over a larger area of tissue, larger amounts of subgenomic polynucleotides or the same amounts re-administered in a successive protocol of administrations, or several administrations to different adjacent or close tissue portions of, for example, a tumor site, may be required to affect a positive therapeutic outcome. In all cases, routine experimentation in clinical trials will determine specific ranges for optimal therapeutic effect.

The therapeutic polynucleotides and polypeptides of the present invention can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., WO 90/07936; WO 94/03622; WO 93/25698; WO 93/25234; U.S. Pat. No. 5,219,740; WO 93/11230; WO 93/10218; U.S. Pat. No. 4,777,127; GB Patent No. 2,200,651; EP 0 345 242; and WO 91/02805), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532), and adeno-associated virus (AAV) vectors (see, e.g., WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. 264:16985 (1989)); eukaryotic cell delivery vehicles cells (see, e.g., U.S. Pat. No. 5,814,482; WO 95/07994; WO 96/17072; WO 95/30763; and WO 97/42338) and nucleic charge neutralization or fusion with cell membranes. Naked DNA can also be employed. Exemplary naked DNA introduction methods are described in WO 90/11092 and U.S. Pat. No. 5,580,859. Liposomes that can act as gene delivery vehicles are described in U.S. Pat. No. 5,422,120; WO 95/13796; WO 94/23697; WO 91/14445; and EP 0524968. Additional approaches are described in Philip, Mol. Cell Biol. 14:2411 (1994), and in Woffendin, Proc. Natl. Acad. Sci. (1994) 91:11581-11585.

Further non-viral delivery suitable for use includes mechanical delivery systems such as the approach described in Woffendin et al., Proc. Natl. Acad. Sci. USA 91(24):11581 (1994). Moreover, the coding sequence and the product of expression of such can be delivered through deposition of photopolymerized hydrogel materials or use of ionizing radiation (see, e.g., U.S. Pat. No. 5,206,152 and WO 92/11033). Other conventional methods for gene delivery that can be used for delivery of the coding sequence include, for example, use of hand-held gene transfer particle gun (see, e.g., U.S. Pat. No. 5,149,655); use of ionizing radiation for activating transferred gene (see, e.g., U.S. Pat. No. 5,206,152 and WO 92/11033).

### Conditions Treatable

Particular aspects of the present invention, for the first time, disclose that Herstatin or Int8 RBD polypeptides, and variants thereof, can not only modulate the expression/level of cellular insulin receptors (IR) (both pro-IR and IR), but also modulate IR-mediated signal transduction (e.g., ERK pathway). According to particular aspects, Herstatin or Int8 RBD polypeptides, and variants thereof can be used in therapeutic methods and pharmaceutical compositions to treat a variety of conditions having an aspect related to, or associated with altered IR expression or altered IR-mediated signaling at a cellular level. Such methods comprising administering to a subject having such a condition, a therapeutically effective amount of a Herstatin or Int8 RBD polypeptide, or a variant thereof, that binds to the extracellular domain of cellular target insulin receptor. Such methods also encompass gene delivery-related methods.

IR is well known in the art to be involved with, inter alia, glycemic control (e.g., hyper- and hypo-glycemia) and glucose metabolism. Accordingly, conditions having an aspect related to, or associated with altered glycemic control and/or glucose metabolism are within the scope of treatable conditions according to the present invention. Such conditions include, but are not limited to insulin resistance syndrome, pre-diabetic conditions, metabolic syndrome, type 1 and type 2 diabetes, cardiac disease, diabetes-associated vascular disease, atherosclerosis, hypertension, diabetes-associated lipid metabolism disorders (dyslipidemia), obesity, critical illness, neurodegenerative disorders, and combinations thereof.

Insulin resistance syndrome has become the major health problem of our times, and is associated with obesity, dyslipidemia, atherosclerosis, hypertension, and type-2 diabetes shorten life spans, and hyperandrogenism with polycystic ovarian syndrome affect quality of life and fertility in increasing numbers of women (see, e.g., Ten & Maclaren, J. Clin Endocrinol Metab., 89:2526-2539, 2004; and see Le Roith 7 Zick, Diabetes Care 24:588-597, 2001; both incorporated herein by reference). In particular preferred aspects, Herstatin or Int8 RBD polypeptide, or variants thereof can be used to treat insulin resistance syndrome.

Insulin resistance and associated abnormalities are believed to have a role in pregnancy induced hypertension (new-onset hypertension), and many features of the insulin resistance syndrome are associated with this condition (see, e.g., Seely & Solomon, J. Clin. Endocrinol. Metab., 88:2393-2398, 2003; incorporated herein by reference). According to the present invention, Herstatin or Int8 RBD polypeptide, or variants thereof can be used to treat hypertension and new-onset hypertension.

In prolonged critical illness neuroendocrine changes lead to more extensive metabolic changes. For example, insulin resistance and hyperglycemia are associated with critical illness (e.g., in surgically critically ill populations with or without diabetes, post-myocardial infarction in patients with diabetes, etc.) (see, e.g., Ronbinson & H. van Soeren, AACN Clinical Issues, 15:45-62, 2004; incorporated herein by reference). According to the present invention, Herstatin or Int8 RBD polypeptide, or variants thereof can be used to treat critical illness.

Significantly, impairment of insulin signaling in the brain has been linked, on the basis of studies using IR-knockout (NIRKO) mice, to neurodegenerative diseases. NIRKO mice exhibit a complete loss of insulin-mediated activation of phosphatidylinositol 3-kinase and insulin-mediated inhibition of neuronal apoptosis, resulting in markedly reduced phosphorylation of Akt and GSK3 β and leading to a substantially increased phosphorylation of the microtubule-associated protein Tau, a hallmark of neurodegenerative diseases (e.g., Alzheimer's disease) (see, e.g., Schubert et al., PNAS 101:3100-3105, 2004, incorporated herein by reference). According to the present invention, Herstatin or Int8 RBD polypeptide, or variants thereof can be used to treat to neurodegenerative diseases (e.g., Alzheimer's disease).

### Combination Therapies

According to additional preferred aspects of the invention, Herstatin-related treatment of conditions having an aspect related to, or characterized by altered glycemic control and/or glucose metabolism, including, but not limited to insulin resistance syndrome, pre-diabetic conditions, metabolic syndrome, type 1 and type 2 diabetes, cardiac disease, diabetes-associated vascular disease, atherosclerosis, hypertension, diabetes-associated lipid metabolism disorders (dyslipidemia), obesity, critical illness, and combinations thereof, may further comprise administration of another therapeutic agent.

For example, the inventive treatment methods may further comprise administering a therapeutically effective amount of a receptor-specific antibody that binds to the extracellular domain of a target receptor selected from the group consisting of: IR, EGFR (HER-1, erbB-1); εEGFR; HER-2 (erbB-2); HER-3 (erbB-3); HER-4 (erbB-4), and IGF-IR.

Alternatively, the inventive treatment methods may further comprise administering a therapeutically effective amount of an agent selected from the group consisting of: insulin, insulin-sensitizing agents, insulin secretogogues, and combinations thereof. Preferably, the insulin-sensitizing agent is selected from the group consisting of biguanides, metformin, thiazolidinediones (glitazones), and combinations thereof. Preferably, the insulin secretogogue is selected from the group consisting of sulfonylureas, meglitinides, and combinations thereof (see, e.g., Zangeneh et al., Mayo Clin Proc., 78:471-479, 2003, incorporated by reference herein).

The present invention will now be illustrated by reference to the following examples which set forth particularly advantageous embodiments. However, it should be noted that these embodiments are illustrative and are not to be construed as restricting the claimed invention in any way.

### EXAMPLE I

### Materials and Methods

### Cell Lines, Transfections, Expression Vectors, Western Blots and Antibodies

Cell lines. IRA-3T3 (3T3 cells transfected with a human insulin receptor cDNA have been previously described (Faria et al., J. Biol. Chem. 269:13922-13928 (1994)), and Herstatin-expressing MCF-7 cell clones were obtained using previously described methods (Shamieh et al., FEBS Letters, 568:163-166, 2004).

Transfections. For transient transfections, 2 μg of empty vector or 2 μg expression vector are added with Lipofectamine™ (GIBCO-BRL) to cells in 6 cm plates.

Western blot analysis, and antibodies. For Western blot analyses, whole-cell lysates or immunoprecipitated proteins were resolved by SDS-PAGE and transferred onto nitrocellulose membranes (BioRad, Hercules, Calif.). Blots were blocked in 5% milk and incubated with primary antibody overnight at 4° C. The antibodies included anti-insulin receptor (IR; against the β subunit), anti-IGF-IR, anti-IRS-1, anti-IRS-2, anti-phosphotyrosine, anti-phospho-Akt, anti-Akt, anti-phospho-ERK, anti-ERK, and anti-Shc antibodies (Santa Cruz Biotechnology, Transduction Laboratories, Cell Signaling Technologies, Upstate Laboratories, or Biosource). After washing, the blots were incubated with secondary antibody conjugated to HRP for 30 min (BioRad, Hercules, Calif.). The membranes were developed with SuperSignal™ West Dura (Pierce, Rockford, Ill.) and exposed to x-ray film.

Expression and Purification of Intron 8-encoded Peptide (Int8) and Herstatin:

Receptor binding domain (RBD). Intron 8 cDNA, in the pET 30 bacterial expression vector (Novagen , Madison, Wis.), is expressed in bacteria (BL-21), and purified by nickel affinity chromatography as described (Doherty et al., supra).

Herstatin. For purification of insect Herstatin, S2 insect cells, stably transfected with 6×His tagged-Herstatin in the pMT/BiP expression plasmid (Invitrogen, Carlsbad, Calif.), were induced with 100 μM cupric sulfate for about 16 hrs. Herstatin was purified to about 90% purity by Ni-NTA (Qiagen, Valencia, Calif.) affinity chromatography as previously described (Jhabvala-Romero et al. Supra.).

### Cell Binding Studies:

ELISA. Monolayer cultures of ˜2×10⁶ cells were plated in 6-well tissue culture plates, and were incubated with purified Herstatin for 2 hours at 4° C. in serum-free DMEM. Cells were washed with Phosphate Buffered Saline (PBS) and extracted in 50 mM Tris·HCl, pH 7.0, 1.0% NP-40. Herstatin bound to cells were quantified using a sandwich Herstatin ELISA per manufacturer's instructions (Upstate Biotechnology, Lake Placid, N.Y.).

The dissociation constant (K_{D}) and maximal binding (Bₘₐₓ) of Herstatin were determined by nonlinear regression analysis of the plot of pmol of bound versus nM of Herstatin added. Statistical comparisons between different binding curves were performed by extra sums-of-squares F-test nonlinear regression coefficients. All tests were performed (α=0.05) using GraphPad™ Prism 4™ software (GraphPad™ Software, 1994-2003).

Pull-down Assays with Int8 Peptide Immobilized on Protein S Agarose:

About 100 μl of a 50% suspension of S-protein agarose (Novagen) is incubated with or without 100 μg of int8 peptide with an S-protein tag, at room temperature for 1 hr, and then washed twice with 500 μl PBS. The agarose samples are then incubated at room temperature for 1 hr with 200 μg of transfected cell extract, then washed twice with 500 μl of PBS with 1% NP40. The proteins associated with the resin are eluted at 92° C. for 2 min in 40 μl of SDS-sample buffer, and analyzed as a Western blot.

### Growth Assays:

Cells (4×10⁴) were plated in quadruplicate in 24-well plates, incubated in serum-free DMEM for 24 hours, and treated with either 10 nM insulin (Sigma) or an equivalent volume of vehicle (25 mM HEPES). At the indicated time points, cell monolayers were washed with PBS and incubated for 30 minutes at 37° C. with 30 μl of MTS reagent [3-(4,5-dimethylthiazol-2-yl)-5-(3-carboxymethoxyphenyl)-2-(4-sulfophenyl-2H-tetrazolium) inner salt Aqueous One Solution (Promega; Madison, Wis.) dissolved in 270 ml PBS] per well. Absorbance at 490 nm was determined a Bio-Tek plate reader.

### EGFR Inhibitor Studies

Control MCF-7 cells were serum-starved overnight and treated with the EGFR kinase inhibitor AG1478 (Sigma) or vehicle (DMSO) for 5 minutes prior to the addition of 14 nM EGF or 10 nM insulin (Sigma). After growth factor treatment, cell lysates were prepared and analyzed for ERK and Akt/PKB activation as described above. The 24-hour treatment was done in regular growth medium.

### EXAMPLE II

### Herstatin was Shown to Bind Specifically to Insulin Receptor (IR) with nM Binding Affinity

The interaction of Herstatin with IR in transfected 3T3 cells (IRA-3T3) was investigated. Herstatin bound specifically to IR at nM concentrations, and IR was thus shown herein to be a target of Herstatin.

Methods. Cell lines, expression vectors, protein purification, pull down assays, antibodies, Western blot analysis and ELISA assays were as described under EXAMPLE I, herein above.

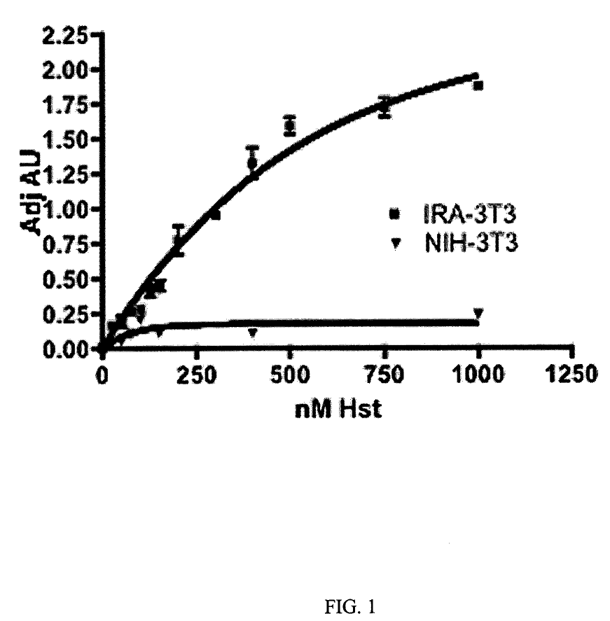

Results. The interaction between Herstatin and IR was investigated. FIG. 1 shows that Herstatin, purified from transfected S2 insect cells, exhibited dose-dependent binding to IR at nM concentrations. Increasing concentrations of Herstatin, expressed and purified from stably-transfected S2 insect cells, were added to 3T3 parental cells (filled triangles; “NIH-3T3”) or 3T3 cells transfected with a human IR cDNA (filled squares; “IRA-3T3”) as previously described (Shamieh et al., FEBS Letters, 568:163-166, 2004). After incubation for 2 hrs on ice, the cells were washed twice with PBS, and the bound Herstatin was quantified using a Herstatin ELISA (Upstate). The data are plotted as Herstatin ELISA units versus concentration added. The results indicate that Herstatin binds at nM concentrations to cells expressing IR, but not to 3T3 parental cells.

These results demonstrate that Herstatin binds specifically to IR with nM binding affinity and that IGF-IR is a target of Herstatin.

### EXAMPLE III

### Herstatin Up-regulated Insulin Receptor (IR) Expression, and Activation of IR by Insulin in MCF-7 Cells

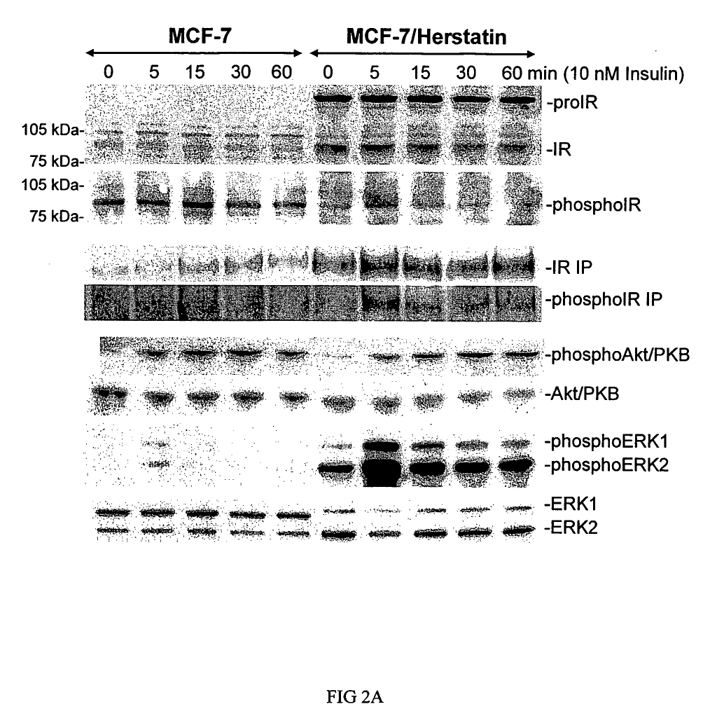

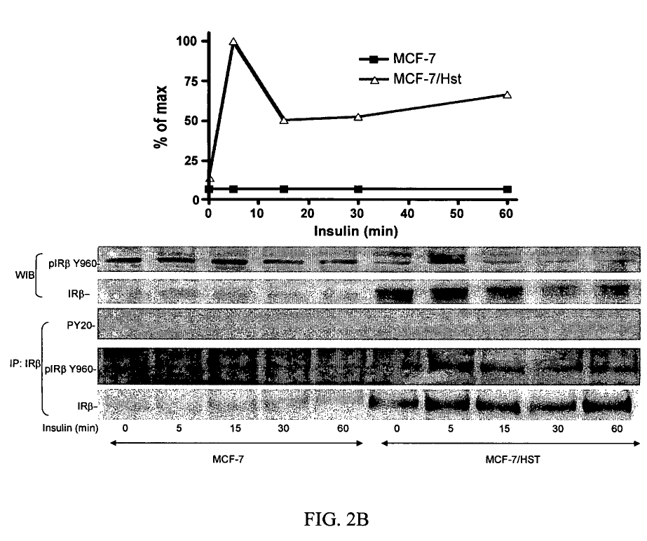

According to particular embodiments of the present invention, Herstatin not only up-regulates IR expression, but also up-regulates activation of IR by insulin (FIG. 2).

Methods. Cell lines, expression vectors, protein purification, pull down assays, antibodies, Western blot analysis and ELISA assays were as described under EXAMPLE I, herein above. Insulin was added either to MCF-7 breast carcinoma cells, or to an MCF-7 cell line stably transfected with a Herstatin expression vector, to determine whether Herstatin expression affects IR expression, and/or insulin-stimulated IR signal transduction.

Results. FIG. 2 shows that Herstatin expression not only up-regulated IR expression (including pro-IR), but also up-regulated IR activation (and thus signaling) in MCF-7 cells. Control and Herstatin-expressing MCF-7 cells were grown in complete medium prior to an overnight incubation in serum-free medium. Insulin was then added to the control and Herstatin-expressing cells and whole-cell lysates were prepared at the indicated times and processed directly for Western immunoblots with anti-insulin receptor (IR), phospho-Akt, Akt, phospho-ERK, and ERK antibodies, or first immunoprecipitated with anti-IR antibody and immunoprecipitates (IP) then analyzed by Western immunoblotting with anti-phosphotyrosine and anti-IR antibodies after transfer to nitrocellulose membranes. Following incubation of blots with primary antibodies, immunoreactive proteins were detected by enhanced chemiluminescence after a secondary incubation with HRP-conjugated secondary antisera. Similar results were obtained with a second Herstatin-expressing MCF-7 clone.

These results demonstrate that Herstatin not only up-regulates IR expression (including pro-IR), but also modulates IR-mediated signaling.

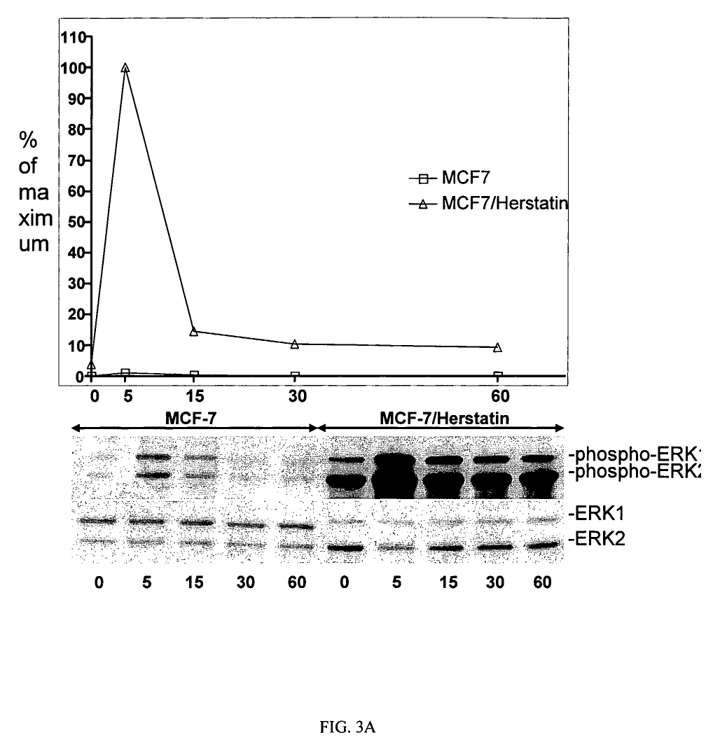

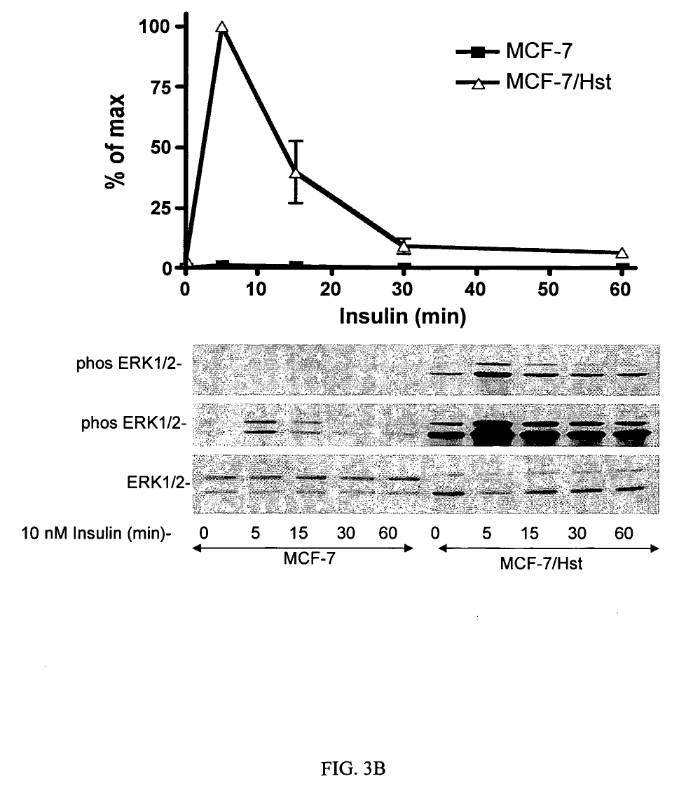

Additionally, as shown in FIG. 2 (see also FIG. 3 below), Herstatin up-regulated insulin-stimulated ERK activation (increased phospho-ERK).

### EXAMPLE IV

### Herstatin Expression Amplified Insulin-stimulated ERK Activation in MCF-7 Cells

The effect of Herstatin expression on insulin-stimulated ERK activation/signaling was further investigated.

Methods. Methods were as described above under EXAMPLE III herein above.

Results. FIG. 3 shows, in MCF-7 cells, that Herstatin expression amplified insulin-stimulated ERK activation. Control and Herstatin-expressing MCF-7 cells were treated and analyzed as those of FIG. 2. Film exposures of enhanced chemiluminescence signals were quantified by scanning densitometry, and the values for the phospho-ERK signals were normalized to the ERK signals to determine the relative level of ERK phosphorylation as a measure of activation.

Herstatin expression substantially amplified insulin-stimulated ERK activation in MCF-7 cells.

According to particular aspects of the present invention, this result supports a substantial utility for Herstatin in treating insulin resistance syndrome, pre-diabetic conditions, metabolic syndrome, type 1 and type 2 diabetes, cardiac disease, diabetes-associated vascular disease, atherosclerosis, hypertension, diabetes-associated lipid metabolism disorders (dyslipidemia), obesity, critical illness, neurodegenerative disorders, and combinations thereof.

This is because the MEK (MAPK kinase)-ERK pathway has been shown to be significantly involved in glucose transport (e.g., Harmon et al., Am. J. Physiol. Endocrinol. Metab., 287:E758-E766, 2004). Specifically, Harmon et al show specific inhibition of MAPK kinase (MEK) by the inhibitors PD-98059 and U-0216, resulting in significant inhibition of insulin-stimulated glucose uptake. The data support the importance of MEK for activation of GLUT4, and further, since the only target of MEK is ERK, the importance of the MEK (MAPK kinase)-ERK pathway for glucose transport.

### EXAMPLE V

### Herstatin Altered the Expression of an Array of Proteins that are Directly Involved in Insulin Action

In addition to the regulation of insulin receptor protein, the regulation of the IRS-1 and IRS-2 proteins and Shc (that function as adapter proteins linking the activated insulin receptor to some of its downstream pathways), the expression of ERK and Akt/PKB, and the regulation of the IGF-IR (which may contribute to enhanced insulin receptor activation by decreasing the proportion of insulin receptor/IGF-I receptor hybrids, which do not respond to insulin) was investigated.

Methods. Cell lines, expression vectors, protein purification, antibodies and ELISA assays were as described under EXAMPLE I, herein above.

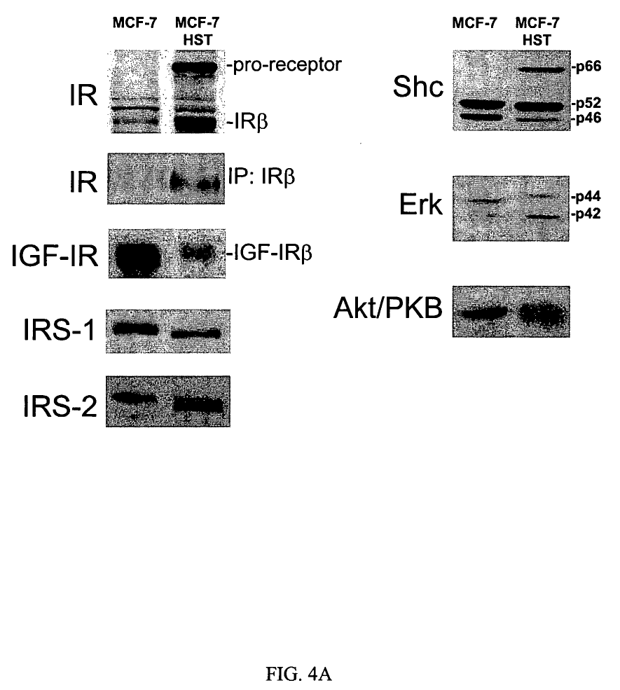

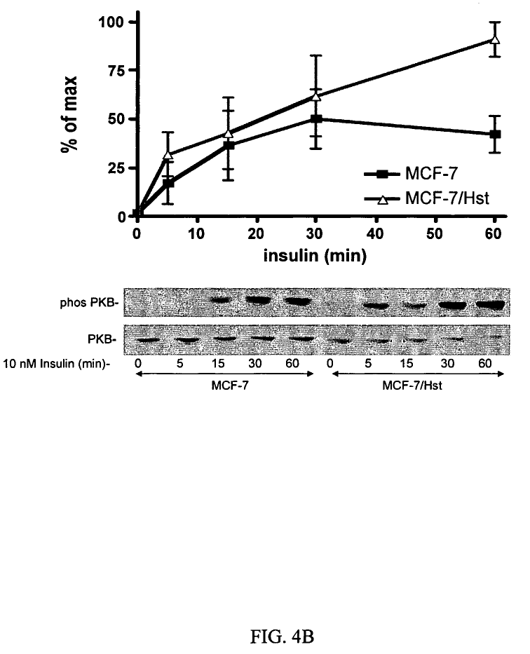

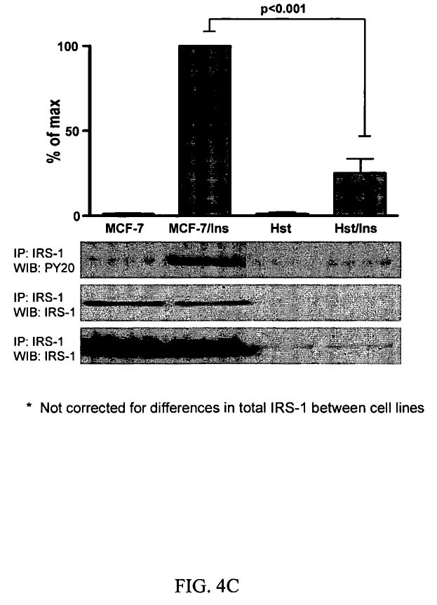

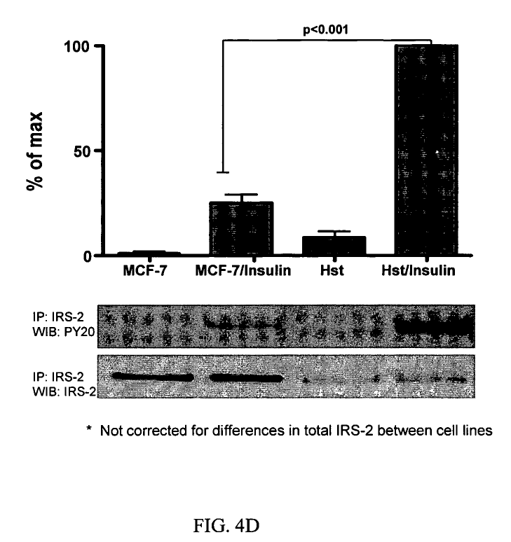

Results. FIG. 4 shows that Herstatin altered the expression of an array of proteins that are directly involved in insulin action. Lysates from control and Herstatin-expressing MCF-7 cells were prepared from respective untreated (no insulin) cells following overnight incubation in serum-free media, and processed directly or (in the case of the IR) also immunoprecipitated prior to Western immunoblot analysis as described in relation to FIG. 2.

These data illustrate that Herstatin: up-regulates insulin receptor protein as assessed by direct Western immunoblot and following immunoprecipitation; mediates the apparent phosphorylation state of the IRS-1 and IRS-2 (differentially down-regulated compared with IRS-1) proteins that function as adapter proteins linking the activated insulin receptor to some of its downstream pathways (see, e.g., Le Roith 7 Zick, Diabetes Care 24:588-597, 2001, discussing role of IRS (IR substrate) proteins in IR-mediated signal transduction); elicits a slight decrease in IRS-2 expression; alters the relative expression of Shc isoforms expressed; increases the relative expression ratio of ERK1 and ERK2; and down-regulates the IGF-IR, which may contribute to enhanced insulin receptor activation by decreasing the proportion of IR/IGF-IR hybrids, which do not respond to insulin.

### EXAMPLE VI

### The EGFR inhibitor AS1478 does not Affect Insulin Signaling or Lead to an Increase in IR

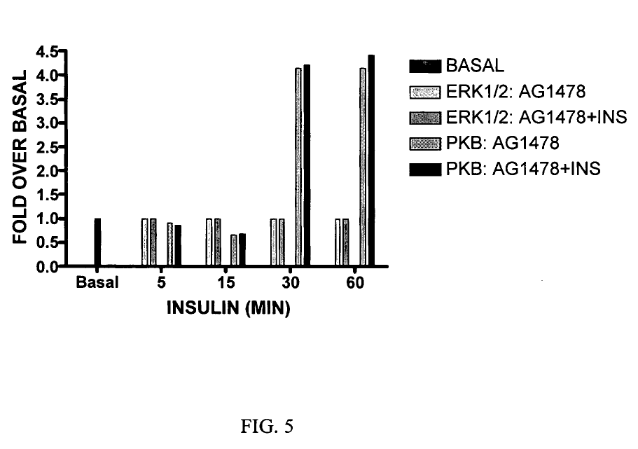

FIG. 5 shows, according to particular aspects, that the EGFR inhibitor AS1478 did not affect insulin signaling.

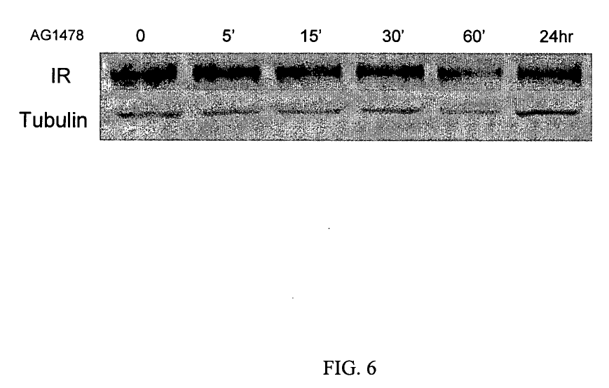

FIG. 6 shows, according to particular aspects, that inhibition of the EGF receptor with an EGF receptor-specific inhibitor did not lead to an increase in insulin receptor.

### OTHER REFERENCES OF INTEREST

Jhabvala-Romero, F., A. Evans, S. Guo, M. Denton, and G. M. Clinton, 2003, Herstatin inhibits heregulin-mediated breast cancer cell growth and overcomes tamoxifen resistance in breast cancer cells that overexpress HER-2: Oncogene, v. 22, p. 8178-86.
Lin, Y. Z., S. W. Li, and G. M. Clinton, 1990, Insulin and epidermal growth factor stimulate phosphorylation of p185HER-2 in the breast carcinoma cell line, BT474: Mol Cell Endocrinol, v. 69, p. 111-9.
Christianson, T. A., J. K. Doherty, Y. J. Lin, E. E. Ramsey, R. Holmes, E. J. Keenan, and G. M. Clinton, 1998, NH2-terminally truncated HER-2/neu protein: relationship with shedding of the extracellular domain and with prognostic factors in breast cancer: Cancer Res, v. 58, p. 5123-9.
Garrett, T. P., N. M. McKern, M. Lou, T. C. Elleman, T. E. Adams, G. O. Lovrecz, H. J. Zhu, F. Walker, M. J. Frenkel, P. A. Hoyne, R. N. Jorissen, E. C. Nice, A. W. Burgess, and C. W. Ward, 2002, Crystal structure of a truncated epidermal growth factor receptor extracellular domain bound to transforming growth factor alpha: Cell, v. 110, p. 763-73.
Filnus, J., M. N. Pollak, J. G. Cairncross, and R. N. Buick, 1985, Amplified, overexpressed and rearranged epidermal growth factor receptor gene in a human astrocytoma cell line: Biochem Biophys Res Commun, v. 131, p. 207-15.
Filmus, J., M. N. Pollak, R. Cailleau, and R. N. Buick, 1985, MDA-468, a human breast cancer cell line with a high number of epidermal growth factor (EGF) receptors, has an amplified EGF receptor gene and is growth inhibited by EGF: Biochem Biophys Res Commun, v. 128, p. 898-905.

1. A method for treating a condition associated with altered insulin receptor expression or altered insulin receptor-mediated signaling, said method comprising administering to a subject in need thereof, a therapeutically effective amount of Herstatin, or a variant thereof, that binds to the insulin receptor.

2. A method for treating a condition associated with altered insulin receptor expression or altered insulin receptor-mediated signaling, comprising administering to a subject in need thereof, a therapeutically effective amount of a Int8 RBD polypeptide, or a variant thereof, that binds to the insulin receptor.

3. The method of any one of claims 1 or 2, wherein the condition is at least one selected from the group consisting of insulin resistance syndrome, pre-diabetic conditions, metabolic syndrome, type 1 and type 2 diabetes, cardiac disease, diabetes-associated vascular disease, atherosclerosis, hypertension, diabetes-associated lipid metabolism disorders (dyslipidemia), obesity, critical illness, and neurodegenerative disorders.

4. The method of any one of claims 1 or 2, wherein the cell further expresses at least one target receptor selected from the group consisting of: EGFR (HER-1, erbB-1); ΔEGFR; HER-2 (erbB-2); HER-3 (erbB-3); HER-4 (erbB-4); and IGF-IR.

5. The method of claim 1, wherein the Herstatin, or variant thereof, comprises a polypeptide selected from the group consisting of SEQ ID NO:2, or a fragment of SEQ ID NO:2 of about 80 to 419 contiguous residues in length, wherein the C-terminal 79 contiguous amino acids are present, wherein at least one N-linked glycosylation site is present, and wherein the polypeptide binds to the insulin receptor.

6. The method of claim 1, wherein the Herstatin, or variant thereof, comprises a sequence selected from the group consisting of SEQ ID NOS:32-42.

7. The method of claim 1, wherein the Herstatin, or variant thereof, comprises SEQ ID NO:32.

8. The method of claim 2, wherein the Int8 RBD polypeptide, or a variant thereof comprises a polypeptide selected from the group consisting of SEQ ID NO:1, or a fragment of SEQ ID NO:1 of about 50 to 79 contiguous residues in length, wherein the polypeptide binds to the insulin receptor.

9. The method of claim 2, wherein the Int8 RBD polypeptide, or a variant thereof, comprises a sequence selected from the group consisting of SEQ ID NOS:21-31,

10. The method of claim 2, wherein the Int8 RBD polypeptide, or a variant thereof, comprises SEQ ID NO:21.

11. The method of any one of claims 1 or 2, further comprising administering a therapeutically effective amount of a receptor-specific antibody that binds to a target receptor selected from the group consisting of: insulin receptor (IR), EGFR (HER-1, erbB-1); ΔEGFR; HER-2 (erbB-2); HER-3 (erbB-3); HER-4 (erbB-4), and IGF-IR.

12. The method of any one of claims 1 or 2, further comprising administration of a therapeutically effective amount of an agent selected from the group consisting of: insulin, insulin-sensitizing agents, insulin secretogogues, and combinations thereof.

13. The method of claim 12, wherein the insulin-sensitizing agent is selected from the group consisting of biguanides, metformin, thiazolidinediones (glitazones), and combinations thereof.

14. The method of claim 12, wherein the insulin secretogogue is selected from the group consisting of sulfonylureas, meglitinides, and combinations thereof.

15. A pharmaceutical composition for treating a condition associated with altered insulin receptor expression or altered insulin receptor-mediated signaling, comprising, Herstatin, or a variant thereof, that binds to the insulin receptor and a pharmaceutically acceptable carrier or excipient.

16. A pharmaceutical composition for treating a condition associated with altered insulin receptor expression or altered insulin receptor-mediated signaling, comprising, a Int8 RBD polypeptide, or a variant thereof, that binds to the insulin receptor and a pharmaceutically acceptable carrier or excipient.

17. The pharmaceutical composition of any one of claims 15 or 16, wherein the condition is selected from the group consisting of insulin resistance syndrome, pre-diabetic conditions, metabolic syndrome, type 1 and type 2 diabetes, cardiac disease, diabetes-associated vascular disease, atherosclerosis, hypertension, diabetes-associated lipid metabolism disorders (dyslipidemia), obesity, critical illness, neurodegenerative disorders, and combinations thereof.

18. The pharmaceutical composition of claim 15, wherein the Herstatin, or variant thereof, comprises a polypeptide selected from the group consisting of SEQ ID NO:2, or a fragment of SEQ ID NO:2 of about 80 to 419 contiguous residues in length, wherein the C-terminal 79 contiguous amino acids are present, wherein at least one N-linked glycosylation site is present, and wherein the polypeptide binds to the insulin receptor.

19. The pharmaceutical composition of claim 16, wherein the Int8 RBD polypeptide, or a variant thereof comprises a polypeptide selected from the group consisting of SEQ ID NO:1, or a fragment of SEQ ID NO:1 of about 50 to 79 contiguous residues in length, wherein the polypeptide binds to the insulin receptor.

20. The pharmaceutical composition of any one of claims 15 or 16, further comprising an agent selected from the group consisting of: insulin, insulin-sensitizing agents, insulin secretogogues, and combinations thereof.

21. The pharmaceutical composition of claim 20, wherein the insulin-sensitizing agent is selected from the group consisting of biguanides, metformin, thiazolidinediones (glitazones), and combinations thereof.

22. The pharmaceutical composition of claim 20, wherein the insulin secretogogue is selected from the group consisting of sulfonylureas, meglitinides, and combinations thereof.

23. A method for targeting a therapeutic agent to a cell expressing insulin receptor, comprising attaching the therapeutic agent to Herstatin, or to a variant thereof, that binds to the extracellular domain of a cellular target insulin receptor.

24. A method for targeting a therapeutic agent to a cell expressing insulin receptor, comprising attaching the therapeutic agent to a Int8 RBD polypeptide, or a variant thereof, that binds to the cellular target insulin receptor.

25. The method of any one of claims 23 or 24, wherein the cell further expresses a target receptor selected from the group consisting of: EGFR (HER-1, erbB-1); ΔEGFR; HER-2 (erbB-2); HER-3 (erbB-3); HER-4 (erbB-4); IGF-IR, and combinations thereof.

26. The method of claim 23, wherein the wherein the Herstatin, or variant thereof, comprises a polypeptide selected from the group consisting of SEQ ID NO:2, or a fragment of SEQ ID NO:2 of about 80 to 419 contiguous residues in length, wherein the C-terminal 79 contiguous amino acids are present, wherein at least one N-linked glycosylation site is present, and wherein the polypeptide binds to the insulin receptor.

27. The method of claim 24, wherein the Int8 RBD polypeptide, or a variant thereof comprises a polypeptide selected from the group consisting of SEQ ID NO:1, or a fragment of SEQ ID NO:1 of about 50 to 79 contiguous residues in length, wherein the polypeptide binds to the insulin receptor.
